(19) United States
(12) Patent Application Publication
(10) Pub. No.: US 2004/0185057 A1
(43) Pub. Date: Sep. 23, 2004

(54) Therapeutical vaccination

(76) Inventors: Nikolai Soren Kirkby, Copenhagen (DK); Kristian Dalsgaard, Kalvehave (DK)

Correspondence Address: BIRCH STEWART KOLASCH & BIRCH, PO BOX 747, FALLS CHURCH, VA, 22040-0747, US

(21) Appl. No.: 10/480,534

(22) PCT Filed: Jun. 14, 2002
(86) PCT No.: PCT/DK02/00404

(30) Foreign Application Priority Data
Jun. 15, 2001 (DK) PA200100939
Jun. 25, 2001 (US) 60300095

(51) Int. Cl. A61K 39/00

(52) U.S. Cl. 424/185.1

(57) ABSTRACT

The present invention describes a therapeutic approach by which malignant or other diseased tissues are at least partly eliminated or removed by action of the diseased individuals own immune system. Provided that the diseased tissue is a cancer the present invention relates to the field of cancer immunotherapy. The basic principle of the invention relies on the establishment of an immune response in the diseased individual against a selected antigen. This is followed by the transfer of the antigen to the diseased cells of the individual by which the elicited immune response is directed against the diseased cells whereby the diseased tissue is eliminated. The immunogen used to induce the immunological response may be, but is not required to be, identical to the antigen. The immuneresponse may exist prior to treatment due to natural infections or may be established by vaccination or by a combination hereof. However, for some applications the active immune component may be provided from heterologous sources and transferred to the individual undergoing treatment e.g. passive transfer of antibodies obtained from another individual or animal or by means of recombinant technology.

### FIELD OF THE INVENTION

The present invention relates to the field of therapeutical vaccination. In particular the present invention is concerned with compounds and methods useful for raising an immune response in an individual followed by introducing foreign antigens to diseased cells of the individual.

### BACKGROUND OF THE INVENTION

During recent years the idea has emerged that apart from using vaccines as a prophylactic measure the accumulating basic knowledge about how vaccines work has resulted in a considerable amount of research into the possibility of using vaccines as therapeutic intervention medicine. This concept has mainly acquired some momentum with the understanding of how cells of the immune system can aquifer a potential for killing other cells through apoptotic signals.

This killing is now well-known in connection with acute infections, but recent experiments has raised the hope that not only infected cells can be destroyed, but that in principle any cell, such as for example malignant cells, which can be specifically recognised by immune cells, may be killed by these immune cells. The deliberate generation of such immune cells is of course a risky enterprise in as much as the immune cells should be able to discriminate between the specific cells and normal cells.

Such procedure may accordingly lead to establishment of a state of autoimmunity. Therefore the major amount of research has concentrated on identifying antigenic determinants naturally associated with cancer cells that make them different from normal cells. Most of this research has been concerned with determining peptides presented on the surface of and specific to cancer cells by major histocompatibility (MHC) class I molecules. Some success using this approach has been achieved recently in a very few cases including humans (melanomas and papilloma virus induced malignancies) particularly when extracorporal in vitro immunisation and subsequent return of these autologous cells has been applied.

The general outcome is however, that even if a cancer-specific MHC class-I antigen has been identified, and even if individuals can be immunised to generate a measurable cytolytic activity against it, the efficiency in vivo is low. The immune system seems to be in a state of anergy in vivo against such antigens. The reason for this is poorly understood, but a plausible explanation could be that since the cancer is de facto established it has developed molecular strategies to obtain acceptance of the body's immune system.

In other words the cancer cells are disregarded or may have developed negative feed back reactions when these antigens are recognised by immune cells. If this is an inherent feature of cancer cells future vaccines against such antigens will have to elicit very strong immune responses to be successful. This again will increase the possibility of “over reactions” and autoimmunity.

In general, therapy against cancer based on the elimination of the malignant cells by mechanisms of the immune system can be referred to as cancer immunotherapy. Cancer immunotherapy is an alternative to the presently applied therapies including surgery, chemotherapy, and radiotherapy. This field has gained much interest in recent years and a multitude of different strategies have been proposed. In general, these approaches are based on immunological responses against the tumour cells by the diseased individuals own immune system, similar in character to the processes engaged for the elimination of infectious agents and in autoimmune diseases.

The anti-tumour immune response may however also be provided by immunologically active cells of heterologous origin. Also, antibodies obtained from serum or antibodies produced in cell culture have been investigated for immune mediated elimination of cancer targets.

The infiltration of cancers by immune active cells has been observed in many naturally occurring solid tumours indicating the ability of the immune system to recognize and react against the malignant tissue. This has motivated the search for suitable tumour-associated antigens (TAA's) against which a strong and curative anti-tumour response can be raised by vaccination. The TAA's are however encoded on the malignant cells chromosome and the structure of the TAA's are either identical to or closely related to self-antigens to which the patient might be expected to be tolerant.

Several approaches to break the tolerance have been investigated including peptide vaccines, DNA-vaccination, infection with recombinant viruses carrying TAA's (Wang et al. 1995, J. of Immunology) etc. Methods utilizing dendritic cells (DC's) primed in vivo with TAA's for subsequent antigens presentation in vivo, as reviewed by Ried (British Journal of Hematology, 2001), have been among the most successful. Sophisticated strategies has been used of priming of DC's including pulsing with whole tumour cell lysates (Knight et al. 1985, PNAS; Grabbe et al. 1995, Immunology Today), defined tumour peptides (Mayordomo et al. 1995, Nature Medicine), acid eluted peptides from tumour MHC molecules (Zitvogel et al. 1996, J. of Experimental Medicine), transfection of DC's with RNA/DNA encoding tumour antigens (Specht et al. 1997, J. of Experimental Medicine) or fusion of DC's with tumour cells (Gong et al. 1998, PNAS).

It remains however unknown if the lack of responsiveness to tumour antigens in naturally occurring diseases is the result of true anergy or failure to recognize the TAA's, which may be of crucial importance for the effectiveness of therapy. Experiments indicate that the very clustering of cells into the form of a solid tumour disables immune surveillance of tumour cells expressing an immunogenic model TAA foreign to the animal (Ochsenbein, 1999, PNAS), supporting the “failure to recognize” theory.

On the other hand even strong cytotoxic responses against TAA's has been observed to abate within limited time. This has been demonstrated in an animal transgenic with respect to both a tumour induction gene and a model self-antigen/TAA, expressed only by tumour cells. Being located on the genome the antigens is a self-antigen despite it viral origin, being restricted to tumour cells the antigen is a TAA. Following infection with the virus from which the antigen was derived, strong cytotoxic responses were induced and anti-tumour immunity observed. However, despite the constitutive expression of the antigen by the tumour cells cytotoxicity declined rendering prolonged tumour suppression dependent on subsequent restimulations of the anti-viral response (Speiser et al., 1997, J. Experimental Medicine). These observations indicate the need for a series of specific antigens to be used sequentially in clinical anti-tumour vaccination.

The inherited risk of inducing autoimmune responses after vaccination with a broad range of antigens must be taken into consideration for such strategies, as this undesirable effect has been observed in an animal model (Roskrow et al. 2000, Leukemia Research).

Induction of specific cytotoxic T-cells has been observed after vaccination with TAA's despite antigen was delivered exogenonusly. This cross-presentation (U.S. Pat. No. 5,951,975) together with the phenomena of epitope spreading (Disis et al., 1999, Clinical Cancer Research; Tary-Lehmann et al., 2000, FASEB Journal), which enables the induction of specific responses against antigen fragments not included in the cancer vaccines may be important factors for the success of the therapy. However, these mechanisms are poorly understood and may limit the number of TAA's useful for anti cancer vaccination.

T-cell therapy is an alternative to the above mentioned ant-cancer vaccination approach. Priming of cytotoxic T-cells is performed in vivo followed by the transfer of these immune cells to the diseased individual, reviewed by Knutson (Journal of Mammary Bland Biology and Neoplasia, 1999). Several methods for the stimulation of T-cells have been proposed and investigated for a range of TAA's in combination with antigen presenting cells and in the presence different cytokines. Although the site of antigen presentation and stimulation of the immunological active cells are different compared to the use of pulsed DC's (in vivo vs. ex vivo) many of the same limitations regarding the selection of useful antigens exist. However these methods may play an essential role for the treatment of diseases such as Hodgkin Disease (U.S. Pat. No. 5,962,318).

Disclosures made by Ogg et al. (British Journal of Cancer, 2000, vol. 82, 5, pp. 1058-1062), WO 01/12223, and WO 01/26681 do not disclose therapeutical vaccination involving the establishment of an immune response in an individual against a selected antigen, followed by transfer of said antigen to diseased cells of the individual, whereby the elicited immune response directed towards said same diseased cells results in the diseased cells being at least partly eliminated.

### SUMMARY OF THE INVENTION

The present invention describes a therapeutic approach by which malignant or other diseased tissues are at least partly eliminated or removed by action of the diseased individuals own immune system. Provided that the diseased tissue is a cancer the present invention relates to the field of cancer immunotherapy.

The basic principle of the invention relies on the establishment of an immune response in the diseased individual against a selected antigen. This is followed by the transfer of the antigen to the diseased cells of the individual by which the elicited immune response is directed against the diseased cells whereby the diseased tissue is eliminated.

The immunogen used to induce the immunological response may be, but is not required to be, identical to the antigen. The immuneresponse may exist prior to treatment due to natural infections or may be established by vaccination or by a combination hereof. However, for some applications the active immune component may be provided from heterologous sources and transferred to the individual undergoing treatment e.g. passive transfer of antibodies obtained from another individual or animal or by means of recombinant technology.

The antigen can be obtained from a wide variety of biological sources or can be synthetically produced. This is a marked feature of the present invention, as the antigen is not dependent on the given disease being treated. Preferably the antigen will be selected as a non-self antigen with respect to the individual undergoing treatment.

The almost unlimited number of possible antigens usable for the treatment of the diseased individual allows for the therapy to be repeated several times, each time using a new antigen. The immunogen is required to establishing the immune response against the antigen, and the same high number of immunogens exists.

The use of an antigen, and a corresponding immunogen, that is not associated with the diseased cells has several advantages:

i) The antigen can be any compound recognisable by the immune system.

ii) Strong immune responses against the antigen can be raised.

iii) The character of the immunological response induced can be controlled.

iv) The risk of inducing autoimmune complications is low.

v) The duration of the action against the targeted cells can be controlled.

vi) The heterogeneity within the population of the diseased cells may not influence presentation of the antigen to the immune system, compared to what has been observed for tumour-associated antigens.

vii) The induction of the immunological response prior to transfer of antigen to the targeted cells reduces the risk of bringing cytotoxic cells into a state of anergy.

viii) A vide variety of delivery technologies can be applied for the transfer of antigen to the targeted cells.

ix) Failure of antigen presentation by cellular processes in the diseased cells may be overcome by selecting antigens not requiring antigen presentation.

Accordingly, it is a first objective of the present invention to provide a kit of parts comprising

i) A foreign immunogen; and

ii) A targeting complex comprising a targeting vehicle capable of being targeted to target cells of an individual, wherein the target cells are desirable to target in order to treat a clinical condition, and a foreign antigen, which can be recognised by an immune response raised against the immunogen.

It is a second objective of the present invention to provide pharmaceutical compositions comprising the kit-of-parts described by the present invention, together with pharmaceutical acceptable carriers.

It is a third objective of the present invention to provide methods of treatment of a condition, which is characterised by the presence of cells capable of being targeted, which are desirable to target in order to treat the clinical condition, in an individual in need thereof comprising the steps of

i) Providing the individual, wherein a protective immune response against a foreign immunogen has been raised; and

ii) Administering a foreign antigen, which is capable of being recognised by the immune response raised against the immunogen; and

iii) Targeting said antigen to the cells of said individual, which are desirable to target; and

iv) Enabling a cytotoxic and/or inflammatory response against said foreign antigen in the individual.

It is fourth objective of the present invention to provide methods of treatment of a condition, which is characterised by the presence of cells, which are desirable to eliminate, in an individual in need thereof comprising administering to said individual the kit-of-parts as described by the present invention, and thereby enabling a cytotoxic and/or inflammatory response against the foreign antigen comprised within the kit-of-parts.

It is a fifth objective of the present invention to provide uses of the kit-of-part described in the present invention, together with a pharmaceutically acceptable carrier for the preparation of a medicament for the treatment of a condition, which is characterised by the presence of cells, which are desirable to target and/or eliminate.

It is a further objective of the present invention to provide methods of non-invasive surgery.

### LEGEND TO FIGURES

FIG. 1 illustrates variants of triterpene aglycones.

### DETAILED DESCRIPTION OF THE INVENTION

Foreign Immunogen and Foreign Antigen

Immunogens according to the present invention are components against which it is possible to raise an immune response in an individual or components, wherein the products of said components can give rise to an immune response. To obtain the immune response it may be necessary that the immunogen is comprised within a vaccine formulation.

Antigens according to the present invention are components, which can be recognised by an immune response, components, wherein fragments of the components can be recognised by an immune response or components, wherein products of said components or fragments of products of said components can be recognised by an immune response.

A foreign immunogen or antigen according to the present invention is an immunogen or an antigen, which is not naturally associated with the condition, which is desirable to treat according to the present invention (see herein below). Accordingly, immunogens or antigen, which are not present within cells or associated with cells, which are desirable to target in order to treat a specific condition, are to be considered as foreign immunogens or antigens in respect of that particular condition.

Preferably, foreign immunogens or antigens are immunogens or antigens, which are not derived from the species, which is to be treated. However, in particular embodiments of the present invention the foreign immunogen or foreign antigens may be derived from the species, which is to be treated. In these embodiments, the immunogen or antigen should not be naturally associated with the cells, which are desirable to target and/or eliminate.

In one preferred embodiment, the foreign immunogen or foreign antigen is not derived from a human being, such as the immunogen or antigen will not be considered as a self-immunogen, when it is administrated to a human being.

In one embodiment of the present invention the foreign immunogen and/or foreign antigen comprises a polypeptide or a peptide, for example the foreign immunogen and/or the foreign antigen may essentially consist of or consist of a polypeptide or a peptide. The immunogen or antigen may also comprise more than one different polypeptide and/or peptide, such as 2, for example 3, such as 4, for example 5, such as 6, for example 7, such as 8, for example 9, such as 10, for example more than 10 different polypeptides.

In some embodiments of the present invention, the foreign immunogen or foreign antigen comprises or essentially consists of an organism, preferably a microorganism or part of an organism, preferably a microorganism and accordingly the immunogen or antigen may comprise a very large number of different polypeptides, such as more than 100, for example more than 500, such as more than 1000, for example more than 2500.

It is also contained within the present invention that foreign immunogens or foreign antigens may essentially consist of or consist of one or more polypeptides and/or peptides. In such embodiments it is preferred that the immunogen is associated with a targeting vehicle (see herein below).

In order to raise an immune response or to enabling an immune response the polypeptides may be processed into fragments and the fragments of the polypeptides may be the compounds, which are actually recognised by the immune response.

Polypeptides according to the present invention may furthermore comprise posttranslational modifications, such as for example phosphorylation, acetylation, methylation, glycosylation or any other posttranslational modification. In particular, in one embodiment of the present invention the foreign immunogen and/or antigen may comprise a glucosylated polypeptide and/or peptide.

In one preferred embodiment of the present invention the foreign immunogen and/or the foreign antigen comprises a lipopeptide, such as a peptide or a polypeptide chemically linked to a lipid moiety, for example the foreign immunogen and/or the foreign antigen may essentially consist of or consist of a peptide or a polypeptide chemically linked to a lipid moiety.

In another embodiment of the present invention the foreign immunogen or foreign antigen comprises a nucleic acid sequence, for example the foreign immunogen or foreign antigen may essentially consist of or consist of a nucleic acid sequence. The immunogen or antigen may comprise more than one different nucleic acid sequence, such as 2, for example 3, such as 4, for example 5, such as 6, for example 7, such as 8, for example 9, such as 10, for example more than 10 different nucleic acid sequences. In some embodiments the immunogen or antigen may essentially consist of or consist of one or more nucleic acid sequences.

Preferably, the nucleic, acid sequences may encode a polypeptide and/or peptide. When the nucleic acid sequence encodes a polypeptide and/or a peptide, preferably, the polypeptide and/or peptide and/or fragments thereof constitute the compound, which is recognised by the immune response.

Accordingly, the following scenario may take place:

i) Nucleic acid sequences are targeted to the target cell

ii) Nucleic acid sequences are internalised into the target cell

iii) Polypeptides and/or peptides are produced within the target cell

iv) Polypeptides and/or peptides and/or fragments thereof are displayed at the cell surface

Preferably, the polypeptides and/or peptides which are comprised within the foreign immunogen or which are encoded by nucleic acid sequences comprised within the immunogen are foreign to the human body. Preferably, the polypeptides and/or peptides which are comprised within the foreign antigen or which are encoded by nucleic acid sequences comprised within the antigen are foreign to the human body.

In yet another embodiment of the present invention the foreign immunogen and or the foreign antigen comprises a polysaccharide and/or oligosaccharide. Polysaccharides and oligosaccharides comprise at least two monosaccharides, which may be identical or different. The empirical formula of a monosaccharide is (CH₂O)ₙ and range in size from trioses (n=3) to heptoses (n=7). Polysaccharides within the scope of the present invention may also be branched polysaccharides.

In one preferred embodiment of the present invention the foreign immunogen or foreign antigen is derived from a virus. In another preferred embodiment of the present invention the foreign immunogen or foreign antigen is derived from a bacteria. In yet another preferred embodiment of the present invention the foreign immunogen or foreign antigen is derived from a parasite. However, the foreign immunogen or foreign antigen may also comprise a mixture of one or more selected from the group consisting of viruses, bacteria and parasites.

The foreign immunogen or foreign antigen may for example be an attenuated virus, bacteria and/or parasite. Alternatively, the foreign immunogen or foreign antigen may be an inactivated or killed microorganism selected from the group consisting of viruses, bacteria and parasites. Mixtures thereof is also contained within the present invention.

Attenuation may for example be accomplished by selecting mutants that have lost pathogenicity after being cultivated for several generations in an unnatural host or after mutagenesis or by manipulation of the microorganism using recombinant DNA techniques. Any other suitable method known to the person skilled in the art may also be used for attenuation.

Inactivation and/or killing of microorganisms may be accomplished by a number of methods, for example heat inactivation, irradiation, chemical inactivation or any other method known to the person skilled in the art.

The foreign immunogen or foreign antigen may furthermore comprise only a part of a microorganism selected from the group consisting of viruses, bacteria and parasites. For example such a part may be a viral capsid. Alternatively, the foreign immunogen or foreign antigen may only comprise one or more molecules, which have been derived from viruses, bacteria and parasites, such as for example polypeptides, peptides or nucleic acid sequences.

Furthermore, the foreign immunogen or foreign antigen may comprise molecules such as for example polypeptides, peptides or nucleic acid sequences, which comprise only fragments of viral, bacterial and parasite derived polypeptides, peptides or nucleic acid sequences. Such molecules may comprise more than one fragment. Such molecules may also be chimeric, such as they in addition comprise fragments which are not derived from a viruses, bacteria and/or parasites or fragments which are derived from another virus, bacteria and/or parasite.

Additionally, the polypeptides, peptides or nucleic acid sequences derived from viruses, bacteria and parasites may have been manipulated, for example using recombinant DNA techniques, such as the polypeptides, peptides or nucleic acid sequences are not the naturally occurring molecules, but rather derivatives or mutants thereof. Mutants include mutants, which comprise substitutions, deletions and/or additions of amino acids or nucleic acids depending on the character of the molecule.

Viruses according to the present invention may for example be selected from the group consisting of: Adeno-associated virus, Adenovirus, Avian infectious bronchitis virus, Baculovirus, Chicken pox, Corona virus, Cytomegaloviruis, Distemper, Enterovirus, Epstein Barr virus, Feline leukemia virus, Flavivirus, Foot and mouth disease virus, Hepatitis A, Hepatitis B, Hepatitis C, Hepatitis E, Herpes species, Herpes simplex, Influenza virus, HIV-1, HIV-2, HTLV 1, Influenza A and B, Kunjin virus, Lassa fever virus, LCMV (lymphocytic choriomeningitis virus), lentivirus, Measles, Mengo virus, Morbillivirus, Myxovirus, Papilloma virus, Parovirus, Parainfluenza virus, Paramyxovirus, Parvovirus, Poko virus, Polio virus, Polyoma tumour virus, pseudorabies, Rabies virus, Reovirus, Respiratory syncytial virus, retrovirus, rhinovirus, Rinderpest, Rotavirus, Semliki forest virus, Sendai virus, Simian Virus 40, Sindbis virus, SV5, Tick borne encephalitis virus, Togavirus (rubella, yellow fever, dengue fever), Vaccinia virus, Venezuelan equine encephalomyelitis and Vesicular stomatis virus.

Preferably, the virus is selected from the group consisting of influenza viruses, herpes viruses, morbili viruses, myxo- and paramyxoviruses, flaviviruses, papillomaviruses and hepatitis viruses.

Bacterias according to the present invention may for example be selected from the group consisting of Achromobacter xylosoxidans, Acinetobacter calcoaceticus, preferably A. anitratus, A. haemolyticus, A. alcaligenes, and A. lwoffii, Actinomyces israelii, Aeromonas hydrophilia, Alcaligenes species, preferably A. faecalis, A. odorans and A. denitrificans, Arizona hinshawii, Bacillus anthracis, Bacillus cereus, Bacteroides fragilis, Bacteroides melaninogenicus, Bordetella pertussis, Borrelia burgdorferi, Borrelia recurrentis, Brucella species, preferably B. abortus, B. suis, B. melitensis and B. canis, Calymmatobacterium granulomatis, Campylobacter fetus ssp. intestinalis, Campylobacter fetus ssp. jejuni, Chlamydia species, preferably C. psittaci and C. trachomatis, Chromobacterium violaceum, Citrobacter species, preferably C. freundii and C. diversus, Clostridium botulinum, Clostridium perfringens, Clostridium difficile, Clostridium tetani, Corynebacterium diphtheriae, Corynebacterium, preferably C. ulcerans, C. haemolyticum and C. pseudotuberculosis, Coxiella burnetii, Edwardsiella tarda, Eikenella corrodens, Enterobacter, preferably E. cloacae, E. aerogenes, E. hafniae (also named Hafnia alvei) and E. agglomerans, Erysipelothrix rhusiopathiae, Escherichia coli, Flavobacterium meningosepticum, Francisella tularensis, Fusobacterium nucleatum, Gardnerella vaginalis, Haemophilus ducreyi, Haemophilus influenzae, Helicobacter species, Klebsiella species, preferably K. pneumoniae, K. ozaenae og K. rhinoscleromatis, Legionella species, Leptospira interrogans, Listeria monocytogenes, Moraxella species, preferably M. lacunata and M. osloensis, Mycobacterium bovis, Mycobacterium leprae, Mycobacterium tuberculosis, Mycoplasma species, preferably M. pneumoniae, Neisseria gonorrhoeae, Neisseria meningitidis, Nocardia species, preferably N. asteroides and N. brasiliensis, Pasteurella haemolytica, Pasteurella multocida, Peptococcus magnus, Plesiomonas shigelloides, Pneumococci, Proteus species, preferably P. mirabilis, P. vulgaris, P. rettgeri and P. morganii (also named Providencia rettgeri and Morganella morganii respectively), Providencia species, preferably P. alcalifaciens, P. stuartii and P. rettgeri (also named Proteus rettgeri), Pseudomonas aeruginosa, Pseudomonas mallei, Pseudomonas pseudomallei, Rickettsia, Rochalimaia henselae, Salmonella species, preferably S. enteridis, S. typhi and S. derby, and most preferably Salmonella species of the type Salmonella DT104, Serratia species, preferably S. marcescens, Shigella dysenteriae, S. flexneri, S. boydii and S. sonnei, Spirillum minor, Staphylococcus aureus, Staphylococcus epidermidis, Staphylococcus saprophyticus, Streptobacillus moniliformis, Streptococcus, preferably S. faecalis, S. faecium and S. durans, Streptococcus agalactiae, Streptococcus pneumoniae, Streptococcus pyogenes, Treponema carateum, Treponema pallidum, Treponema pertenue, preferably T. pallidum, Ureaplasma urealyticum, Vibrio cholerae, Vibrio parahaemolyticus, Yersinia enterocolitica, and Yersinia pestis.

Parasites according to the present invention may for example be selected from the group consisting of Malaria (Plasmodium. falciparum, P. vivax, P. malariae), Schistosomes, Trypanosomes, Leishmania, Filarial nematodes, Trichomoniasis, Sarcosporidiasis, Taenia (T. saginata, T. solium), Leishmania, Toxoplasma gondii, Trichinelosis (Trichinella spiralis) or Coccidiosis (Eimeria species).

The foreign immunogen and/or foreign antigen could furthermore be derived a fungus selected from the group consisting of Cryptococcus neoformans, Candida albicans, Aspergillus fumigatus and Coccidioidomycosis.

It is however possible, that the foreign immunogen and/or the foreign antigen is derived from any animal, including for example vertebrates. For example the foreign immunogen and/or foreign antigen may comprise components derived from or may essentially consist of the group consisting of ovalbumin, keyhole limpet hemocyanin and sperm-whale myoglobulin.

The foreign immunogen and/or the foreign antigen may in one embodiment comprise a hapten linked to a carrier molecule, for example the foreign immunogen and/or the foreign antigen may essentially consist of or consist of a hapten linked to a carrier molecule. Alternatively, the product of the foreign immunogen and/or the foreign antigen may comprise or essentially consist of or consist of a hapten linked to a carrier molecule. Haptens are small chemically defined compounds that become immunogenic upon conjugation to a carrier molecule, which however are only weakly immunogenic alone.

The foreign immunogen and/or the foreign antigen may in another embodiment be a multivalent immunogen and/or antigen. Multivalent antigens and/or immunogens may for example be selected from the group consisting of T-independent type 2 antigens, synthetic polymers and multimeric peptide antigens. However the foreign immunogen and/or the foreign antigen may also be a any other mutivalent antigen and/or immunogen known to the person skilled in the art.

Preferably, the foreign immunogen and/or the foreign antigen comprises a peptide capable of being presented by MHC molecules, for example the foreign immunogen and/or the foreign antigen may essentially consist of or consist of a peptide capable of being presented by MHC molecules. Alternatively, the products of the foreign immunogen and/or the foreign antigen comprises a peptide capable of being presented by MHC molecules, for example the products of the foreign immunogen and/or the foreign antigen may essentially consist of or consist of a peptide capable of being presented by MHC molecules.

The peptide capable of being presented by MHC molecules may be any such peptide known to the person skilled in the art. Preferred examples of such peptides are listed in the following database: “MHCPEP—A database of MHC binding peptides (v. 1.3)”, compiled by Brusic (V. Brusic, G. Rudy, A. P. Kyne and L. C. Harrison; MHCPEP, a database of MHC-binding peptides: update 1997; Nucleic Acids Research, 1998, Vol 26, No. 1, pp. 368-371).

In addition, the foreign antigen and/or the foreign immunogen may comprise a mixture of two or more of the above mentioned foreign immunogens and foreign antigens.

In one preferred embodiment of the present invention the antigen and/or immunogen is a peptide selected for the capability of binding to a MHC molecule. Accordingly, the selected peptide is presented at the cell surface associated with the MHC molecule. Alternatively, the product of the foreign immunogen and/or the foreign antigen is a peptide capable of binding to a MHC molecule. An example of such a peptide antigen/immunogen is a plasmid vector, a linear nucleic acid sequence or a viral vector encoding the peptide under the control of a suitable promoter and as such being expressed in vivo. Such peptide antigens encoded by nucleic acids are often termed minigene nucleic acid vaccines (Fomsgaard, A. et al. 1999, Vaccine 18, 681-691; Ishioka, G. Y. et al. 1999, J. Immunol. 162, 3915-3925).

Each MHC molecule is characterized by its capability to bind a repertoire of peptides and present these at the cell surface to other cells of the immune system, such as e.g. T-cells. Such MHC-binding peptides are often termed peptide epitopes or in short epitopes. A specific MHC molecule binds different peptides with different affinity, however some peptides may bind with the same affinity. Hence, the repertoire of peptides binding a specific MHC molecule can be divided into categories according to the binding affinity of the individual peptides to the MHC molecules. Peptides can be divided into different categories depending on their binding affinity. Peptides can e.g. be divided into three categories termed high affinity binding peptides, intermediate affinity binding peptides, and low affinity binding peptides. However, peptides can also be further divided into even more categories; such as for example 4, 5 or 6 categories. Each category defines a range of binding affinities and peptides are categorized depending on their binding affinity to a specific MHC molecule.

Independent of the number of categories defined, peptides with the highest binding affinity to a specific MHC molecule are, with respect to the present invention, termed high affinity binding peptides, viz. epitopes. This category of peptides is, with respect to the present invention, designated category one (1), whereas the following categories are designated a higher number. This in such a way, that peptides belonging to category two (2) has higher binding affinity to the specific MHC molecule than peptides belonging to category three (3), that has higher binding affinity to the specific MHC molecule than peptides belonging to category four (4), that has higher binding affinity to the specific MHC molecule than peptides belonging to category five (5). Accordingly, peptides belonging to category five (5) have higher binding affinity to the specific MHC molecule than peptides belonging to category six (6). Antigen/immunogen peptides with binding affinity to a specific MHC molecule lower than the range of binding affinities defined for the category of high affinity binding peptides (category 1) are with respect to the present invention termed sub-optimal binding affinity peptides.

The binding affinity of a given peptide to a given MHC molecule can be determined by several different assays. Examples of such assays is described e.g. by Altfeld, M. A. et al., 2001, J. Virol. 75, 1301-1311; Buus, S. et al. 1995, Biochim. Biophys. Acta 1243, 453-460. Peptide binding affinity is often expressed as peptide binding capacity and measured as the IC50 value in nano-molar (nM) for a given peptide and a given MHC molecule. This in such a way that peptides with high binding affinity has a low IC50 value, whereas peptides with low binding affinity has high IC50 value respectively. Examples of different peptide epitopes and their corresponding binding capacity to some selected alleles of human MHC class-I molecules of the HLA-A locus are given in the following table:

Binding capacity of selected antigen/immunogen
peptides to different alleles of the HLA-A2 super type
MHC class-I molecule, expressed as IC50 values in nM.

| | HLA-A2 allele | | | | |
|---|---|---|---|---|---|
| Peptide sequence | A*0201 | A*0202 | A*0203 | A*0206 | A*6802 |
| MTNNPPIPV | 166.6 | 7,166.7 | 33.3 | 1,608.7 | 12.1 |
| VLAEAMSQV | 66.6 | 82.7 | 15.2 | 115.6 | 363.6 |
| KMIGGIGGFI | 172.4 | 54.4 | 4.8 | 770.8 | 3,333.3 |
| LVGPTPVNI | 454.5 | 153.6 | 19.2 | 2,846.2 | 67.8 |
| CTLNFPISPI | 147 | 23.9 | 30.3 | 8.4 | 100 |
| TLNFPISPI | 75.7 | 1,482.8 | 1.1 | 1,947.4 | 57.1 |
| ALVEICTEM | 217.3 | 187 | 140.8 | 264.3 | 2,857.1 |
| YTAFTIPSI | 26.3 | 6.1 | 9.1 | 7 | 16.7 |
| KLVGKLNWA | 59.5 | 12.6 | 5.9 | 39.8 | 3,076.9 |
| RAMASDFNL | 217.3 | 116.2 | 25,000 | 52.1 | 3,076.9 |
| MASDFNLPPV | 62.5 | 22.6 | 55.6 | 33.6 | 18.2 |
| KLTPLCVTL | 102 | 126.5 | 66.7 | 185 | 20,000 |
| LLQLTVWGI | 9.8 | 215 | 43.5 | 24.7 | 645.2 |
| KAACWWAGI | 277.7 | 1,075 | 83.3 | 160.9 | 2,666.7 |
| LTFGWCFKL | 35.7 | 33.1 | 4,545.5 | 205.6 | 5.6 |
| LTFGWCFKLV | 294.1 | 48.9 | 185.2 | 57.8 | 6.2 |
| AIIRILQQL | 333.3 | 22.6 | 41.7 | 38.5 | 547.9 |
| RILQQLLFI | 19.2 | 1,535.7 | 125 | 37 | 1,818.2 |
| SLLNATDIAV | 9.8 | 1,303 | 238.1 | 28.5 | 5,479.4 |
| ILKEPVHGV | 192.3 | 2,388.9 | 6.7 | 37,000 | 363.6 |

Compiled from Altfeld et al., Journal of Virology, 2001, p. 1301-1311

Historically, peptide antigens and/or immunogens with high affinity has been selected for use in both prophylactic and therapeutic vaccine (Altfeld, M. A. et al., 2001, J. Virol. 75, 1301-1311) as it is anticipated that high binding affinity to MHC molecules correlate with efficient presentation of the peptide to T-cells. For several antigen/immunogen peptides, this anticipation has been shown to correlate with the biophysical observations. The obtained advantage of using such highly efficient peptides, viz epitopes, is that very strong immunological responses against the peptide and/or the antigen/immunogen from which the peptide is derived can be obtained. However, only few MHC high affinity binding peptide antigens/immunogens can be obtained compared to the number of peptide antigens/immunogenes with lower MHC binding affinity. In terms of the categories defined above the number of antigen/immunogen peptides belonging to category 1 (high affinity binding peptides) is typically significantly lower than the sum of the peptides belonging the sub-optimal binding affinity peptides, with respect to a selected MHC molecule, independently of the immunogen and/or antigen or group of immunogens and/or antigens from which the peptides are derived from, when the immunogen and/or antigen is a polypeptide significantly longer than the length of peptide required to bind the MHC molecule of interest.

In one preferred embodiment of the present invention, the required length of the immunogen/antigen peptide required for binding to a selected MHC class-I molecules is 6 amino acids or 7 amino acids or 8 amino acids preferentially 9 amino acids or 10 amino acids.

Antigen/immunogen peptides with sub-optimal binding affinity are more frequent than antigen/immunogen peptides belonging to the category of high affinity binding peptides but also allows for phenomena's known as epitope spreading and cross presentation to occur. These phenomena allows for the induction of immunological responses targeted to peptide antigens/immunogens other than the foreign peptide antigen/immunogen. Accordingly, immune responses to self-antigens/self-immunogens can be induced by utilization of sub-optimal binding affinity foreign antigen/immunogen peptides. Preferably, the self-antigen/self-immunogen is a tumour-associated antigen (TAA). In another preferred embodiment the immunological cross-reactivity or epitope spreading phenomena is directed against immunogenic and/or antigenic peptides of non-self origin derived from one or more infectious pathogens, examples are bacteria, virus, parasites and/or fungi, as listed above. The immunogenic and/or antigenic peptides of non-self origin can also be synthetic or synthetically produced by any state of the art technique allowing such synthesis.

In one preferred embodiment, the foreign antigen and/or foreign immunogen comprises a selection of such peptides selected for their binding affinity to specific MHC molecules as described above. Accordingly, the foreign antigen and/or foreign immunogen may comprise e.g. from 2 peptides to and including 5 peptides selected for their binding affinity to one or more MHC molecules, such as e.g. 3 or 4 peptides, or the foreign antigen and/or foreign immunogen may comprise from 6 to and including 10 peptides selected for their binding affinity to one or more MHC molecules, such as 7, 8, or 9 peptides, or it may comprise from between 11 and 25 peptides selected for their binding affinity to one or more MHC molecules, alternatively may comprise more than 25 peptides.

The foreign antigen and/or foreign immunogen peptides may be selected for their binding affinity either a MHC class-I or MHC class-II molecules. In addition the foreign antigen and/or foreign immunogen may comprise both antigen/immunogen peptides selected for their binding affinity to one or more specific MHC class-I molecules and peptides selected for their binding affinity to one or more specific MHC class-II molecules. Alternatively, the foreign antigen and/or foreign immunogen may comprise antigen/immunogen peptides selected for their binding to both one or more MHC class-I molecules and one or more MHC class-II molecules.

In one preferred embodiment, the MHC molecule to which antigen/immunogen peptide binds is a MHC class-I molecule. Examples of such MHC class-I molecules are: HLA-A, HLA-B and HLA-C, including the super types HLA-A2, HLA-A3, HLA-B7, HLA-B27, HLA-B37, including the alleles A*0201, A*0202, A*0203, A*206, A*6802.

In another preferred embodiment, the MHC molecule to which antigen/immunogen peptide binds is a MHC class-II molecule. Examples of such MHC class-II molecules are: HLA-DR, HLA-DQ, and HLA-DP.

In one preferred embodiment of the invention the antigen/immunogen peptides are divided into three (3) categories depended on their binding capacity to a selected MHC class-I molecule. The categories include peptides with high binding capacity of (IC50≦50 nM), intermediate binding capacity (50 nM<IC50≦500 nM), and low binding capacity (IC50<500 nM).

When the peptides belong to the group having a high binding capacity (i.e. within the range of 0 nM<IC50≦50 nM), preferred ranges include for example 0 nM<IC50≦40 nM; 0 nM<IC50≦30 nM, 0 nM<IC50≦20 nM, 0 nM<IC50≦10 nM, 0 nM<IC50≦5 nM; 5 nM<IC50≦50 nM; 10 nM<IC50≦50 nM; 15 nM<IC50≦50 nM; 20 nM<IC50≦50 nM; 25 nM<IC50≦50 nM; 30 nM<IC50≦50 nM; 35 nM<IC50≦50 nM; 40 nM<IC50≦50 nM; and 45 nM<IC50≦50 nM. Additionally preferred ranges are 5 nM<IC50≦10 nM; 10 nM<IC50≦15 nM; 15 nM<IC50≦20 nM; 20 nM<IC50≦25 nM; 25 nM<IC50≦30 nM; 30 nM<IC50 ≦35 nM; 35 nM<IC50≦40 nM; 40 nM<IC50≦45 nM; and 45 nM<IC50≦50 nM.

When the peptides belong to the group having an intermediate binding capacity (i.e. within the range of 50 nM<IC50≦500 nM), preferred ranges include for example 50 nM<IC50≦400 nM; 50 nM<IC50≦300 nM, 50 nM<IC50≦200 nM, 50 nM<IC50≦150 nM, 50 nM<IC50≦125 nM; 50 nM<IC50≦100 nM; 50 nM<IC50≦75 nM; 75 nM<IC50≦500 nM; 100 nM<IC50≦500 nM; 125 nM<IC50≦500 nM; 150 nM<IC50≦500 nM; 200 nM<IC50≦500 nM; 250 nM<IC50≦500 nM; 300 nM<IC50≦500 nM; 350 nM<IC50≦500 nM; and 400 nM<IC50≦500 nM. Additionally preferred ranges are 50 nM<IC50≦100 nM; 100 nM<IC50≦150 nM; 150 nM<IC50≦200 nM; 200 nM<IC50≦250 nM; 250 nM<IC50≦300 nM; 300 nM<IC50≦350 nM; 350 nM<IC50≦400 nM; 400 nM<IC50≦450 nM; and 450 nM<IC50≦500 nM.

When the peptides belong to the group having a low binding capacity (i.e. within the range of IC50>500 nM), preferred ranges include for example IC50>600 nM; IC50>700 nM; IC50>800 nM; IC50>900 nM; IC50 >1000 nM; IC50>1100 nM; IC50>1200 nM; IC50>1300 nM; IC50>1400 nM; IC50>1500 nM; and preferably an IC50 value lower than 2000 nM.

Of special relevance for the present invention are antigen/immunogen peptides of intermediate binding capacity, e.g. 50 nM<IC50≦500 nM for a selected MHC class-I molecule. The selected MHO class-I molecule are for example, but not limited to, HLA-A2, HLA-A3, HLA-B7, HLA-B27, HLA-B37. Accordingly, the foreign antigen/immunogen can comprise a number of such antigen/immunogen peptides as described herein immediately above.

Preferably, the foreign antigen may be recognised by an immune response raised against the foreign immunogen. Accordingly, the foreign antigen and the foreign immunogen should preferably resemble one another.

More preferably, they resemble one another so that they comprise compounds, which alone or together with other molecules are capable of associating with the same interacting molecule. Alternatively, they comprise components, wherein fragments of the components alone or together with one or more other molecules are capable of associating with the same interacting molecule. Interacting molecules within this context are preferably components of the immune system. For example interacting molecules may be selected from the group consisting of antibodies and T-cell receptors.

In one embodiment of the present invention the antigen and the immunogen are the same, meaning that the immunogen and the antigen are identical. In another embodiment of the present invention the antigen is a fragment of the immunogen. In yet another embodiment of the present invention the immunogen is a fragment of the antigen. In a yet further embodiment of the present invention the antigen mimics the immunogen.

The kit-of-parts according to the present invention may comprise more than one immunogen. For example 2, such as 3, for example 4, such as 5, for example 6, such as 7, for example 8, such as 9, for example 10, such as more than 10 different foreign immunogens.

The kit-of-parts according to the present invention may comprise more than one antigen. For example 2, such as 3, for example 4, such as 5, for example 6, such as 7, for example 8, such as 9, for example 10, such as more than 10 different foreign antigen.

Vaccine

The immunogen and/or the antigen according to the present invention may be contained within a vaccine formulation. Any vaccine formulation known to the person skilled in the art may be used with the present invention.

The vaccine formulation may comprise more than one immunogen or antigen, such as 2, for example 3, such as 4, for example 5, such as more than 5 different antigens. The immunogens and antigens may be selected from the group of immunogens and antigens described herein above.

Preferably, the vaccine formulation according to the present invention furthermore comprises an adjuvant. The vaccine formulation according to the present invention may furthermore comprise a carrier. The carrier or adjuvant could be any carrier or adjuvant known in the art including functional equivalents thereof. Functionally equivalent carriers are capable of presenting the same antigen in essentially the same steric conformation when used under similar conditions. Functionally equivalent adjuvants are capable of providing similar increases in the efficacy of the composition when used under similar conditions.

Preferably, said formulations comprise potent, nontoxic adjuvants that will enhance and/or modulate the immunogenicity of immunogenic determinants including antigenic determinants including haptenic determinants represent one group of preferred adjuvants. In addition, such adjuvants preferably also elicit an earlier, more potent, or more prolonged immune response. Such an adjuvant would also be useful in cases where an antigen supply is limited or is costly to produce.

Adjuvants pertaining to the present invention may be grouped according to their origin, be it mineral, bacterial, plant, synthetic, or host product. The first group under this classification is the mineral adjuvants, such as aluminum compounds. Antigens precipitated with aluminum salts or antigens mixed with or adsorbed to performed aluminum compounds have been used extensively to augment immune responses in animals and humans. Aluminium particles have been demonstrated in regional lymph nodes of rabbits seven days following immunisation, and it may be that another significant function is to direct antigen to T cell containing areas in the nodes themselves. Adjuvant potency has been shown to correlate with intimation of the draining lymph nodes. While many studies have confirmed that antigens administered with aluminium salts lead to increased humoral immunity, cell mediated immunity appears to be only slightly increased, as measured by delayed-type hypersensitivity. Aluminium hydroxide has also been described as activating the complement pathway. This mechanism may play a role in the local inflammatory response as well as immunoglobulin production and B cell memory. Furthermore, aluminum hydroxide can protect the antigen from rapid catabolism. Primarily because of their excellent record of safety, aluminum compounds are presently the only adjuvants used in humans.

Another large group of adjuvants is those of bacterial origin. Adjuvants with bacterial origins can be purified and synthesized (e.g. muramyl dipeptides, lipid A) and host mediators have been cloned (Interleukin 1 and 2). The last decade has brought significant progress in the chemical purification of several adjuvants of active components of bacterial origin: Bordetella pertussis, Mycobacterium tuberculosis, lipopolysaccharide, Freund's Complete Adjuvant (FCA) and Freund's Incomplete Adjuvant (Difco Laboratories, Detroit, Mich.) and Merck Adjuvant 65 (Merck and Company, Inc., Rahway, N.J.). Additionally suitable adjuvants in accordance with the present invention are e.g. Titermax Classical adjuvant (SIGMA-ALDRICH), ISCOMS, Quil A, ALUN, see U.S. Pat. Nos. 58,767 and 5,554,372, Lipid A derivatives, choleratoxin derivatives, HSP derivatives, LPS derivatives, synthetic peptide matrixes, GMDP, and other as well as combined with immunostimulants (U.S. Pat. No. 5,876,735).

B. pertussis is of interest as an adjuvant in the context of the present invention due to its ability to modulate cell-mediated immunity through action on T-lymphocyte populations. For lipopolysaccharide and Freund's Complete Adjuvant, adjuvant active moieties have been identified and synthesized which permit study of structure-function relationships. These are also considered for inclusion in immunogenic compositions according to the present invention.

Lipopolysaccharide and its various derivatives, including lipid A, have been found to be powerful adjuvants in combination with liposomes or other lipid emulsions. It is not yet certain whether derivatives with sufficiently low toxicity for general use in humans can be produced. Freund's Complete Adjuvant is the standard in most experimental studies.

Mineral oil may be added to vaccine formulation in order to protect the antigen from rapid catabolism.

Many other types of materials can be used as adjuvants in immunogenic compositions according to the present invention. They include plant products such as saponin, animal products such as chitin and numerous synthetic chemicals.

Adjuvants according to the present invention can also been categorized by their proposed mechanisms of action. This type of classification is necessarily somewhat arbitrary because most adjuvants appear to function by more than one mechanism. Adjuvants may act through antigen localization and delivery, or by direct effects on cells making up the immune system, such as macrophages and lymphocytes. Another mechanism by which adjuvants according to the invention enhance the immune response is by creation of an antigen depot This appears to contribute to the adjuvant activity of aluminum compounds, oil emulsions, liposomes, and synthetic polymers. The adjuvant activity of lipopolysaccharides and muramyl dipeptides appears to be mainly mediated through activation of the macrophage, whereas B. pertussis affects both macrophages and lymphocytes. Further examples of adjuvants that may be useful when incorporated into immunogenic compositions according to the present invention are described in U.S. Pat. No. 5,554,372.

In one preferred embodiment, adjuvants according to the present invention are selected from the group consisting of aluminium compounds, Freunds incomplete adjuvant, Titermax classical adjuvant and oil emulsions.

There is also provided an embodiment of the present invention wherein the vaccine formulation further comprises a carrier. The carrier may be present independently of an adjuvant. The purpose of conjugation and/or co-immunisation of an antigen and a carrier can be e.g. to increase the molecular weight of the antigen in order to increase the activity or immunogenicity of the antigen, to confer stability to the antigen, to increase the biological activity of the determinant, or to increase its serum half-life. The carrier protein may be any conventional carrier including any protein suitable for presenting antigens. Conventional carrier proteins include, but are not limited to, keyhole limpet hemocyanin, serum proteins such as transferrin, bovine serum albumin, or human serum albumin, an ovalbumin, immunoglobulins, or hormones, such as insulin.

The vaccine formulation according to the present invention may furthermore comprise a biological active component. A biological active component may be any component which directly or indirectly can influence the immune response of an individual. Preferably, the biological active component is selected from the group consisting of cytokines and chemokines.

Cytokines may for example be selected from the group consisting of IL-2, IL-4, IL-10, IL-12, IL-15, IL-18, IL-21, IFN-γ, IFN-60 , GM-CSF, C-CSF.

In one embodiment of the present invention the immunogen is associated with a targeting vehicle. Any targeting vehicle as described herein below may be associated with the immunogen. When the immunogen is a polypeptide and/or a peptide the targeting vehicle preferably is selected from the group consisting of posintros, ISCOMs, QS21 and MPL.

Targeting Vehicle

The targeting vehicle according to the present invention may be any targeting vehicle capable of being targeted to target cells of an individual, wherein the target cells are desirable to target and/or eliminate. A number of different targeting vehicles are known to the person skilled in the art and a suitable targeting vehicle may be selected according to the specific need.

In one specifically preferred embodiment of the present invention the targeting vehicle is a posintro or a cationic ISCOM. The terms “posintro and “catinonic ISCOM” are used interchangeably throughout the description.

Posintros or cationic ISCOMS may for example be any of the compounds described in the Danish patent application PA 2001 00560, which is hereby incorporated by reference in its entirety.

Posintros within the scope of the present invention are complexes comprising:

i) at least one first sterol and/or at least one second sterol,

wherein the at least one second sterol is capable of contacting a foreign antigen, preferably a nucleic acid by means of an interaction selected from an electrostatic interaction and a hydrophobic interaction, and

wherein the at least one first sterol and/or the at least one second sterol is capable of forming a complex with at least one first saponin and/or at least one second saponin, and

ii) at least one first saponin and/or at least one second saponin,

wherein the at least one second saponin is capable of contacting a genetic determinant by means of an interaction selected from an electrostatic interaction and a hydrophobic interaction, and

wherein the at least one first saponin and/or the at least one second saponin is capable of forming a complex with at least one first sterol and/or at least one second sterol, and optionally

iii) at least one contacting group for contacting a genetic determinant by means of an interaction selected from an electrostatic interaction and a hydrophobic interaction,

with the proviso that the at least one contacting group is present when no second sterol and no second saponin is present in the complex and further optionally

i) at least one lipophilic moiety.

Posintros according to the present invention may in one preferred embodiment adopt a micro-particle structure in the form of a cage-like matrix similar to that known as an immune stimulating complex (iscom). Beside iscom structures, the interaction between sterols and saponins have been reported to result in a variety of different structural entities, including entities such as e.g. lattices, honeycombes, rods, and amorphic particles, all of which structural entities are covered by the present invention.

Accordingly, a glycoside solution, containing fx cholesterol, phospholipid, and one or more glycosides (fx Quillaja components) with hydrophobic and hydrophilic domains in a concentration of at least a critical micelle-binding concentration, is formed and a complex is generated. The complex may subsequently be isolated and/or purified.

Optionally, as a first step, the component to be inserted into the vehicle for example the immunogen or the antigen, can be mixed with one or more solubilizing agents, whereby complexes are formed between the component and solubilizing agents, after which the components are separated from the solubilizing agent and e.g. transferred directly to the glycoside solution.

In line with the present invention, the glycoside solution may initially be mixed with a polynucleotide. It is possible to proceed from a matrix that can be made by solubilizing at least one sterol in a solution agent, adding at least one glycoside or at least one saponin, and optionally the other lipophilic moieties, after which addition the solution agent may be removed, if it is proving unacceptable to the final product.

The matrix may be transferred to a water solution in which its separate parts are not soluble. The solubilizing agent can be removed through e.g. gel filtration, ultra filtration, dialysis, or electrophores. The matrix can then be purified from surplus of first sterol and saponin e.g. by ultracentrifugation, through a density gradient, or through gel filtration. The solubilizing agent may be any one of those mentioned in U.S. Pat. No. 5,679,354, which is incorporated herein by reference.

Saponins pertaining to the present invention are described in detail herein below. Saponins are glycosidic compounds which comprises an aglycone compound and a saccharide compound linked together by a glycosidic bond. The asymmetric distribution of their hydrophobic (aglycone) and hydrophilic (saccharide) moieties confers an amphipathic character to the saponins according to the present invention.

Saponins are produced by many organisms as secondary metabolites. They are widely distributed among higher plants and in some marine invertebrates. Plant material often contains triterpene saponins in considerable amounts. Thus, primula root contains about 5-10% saponin, licorice root between 2% and 12% glycyrrhizin, quillaja bark up to 10% of a saponin mixture and the seeds of the horse chestnut up to 13% aescine. In other words, the concentration of saponins in plants is high when compared with other secondary metabolites.

The aglycone or non-saccharide portion of the saponin molecule is called the genin or sapogenin. Depending on the type of genin present, the saponins can be divided into three major classes: i) triterpene glycosides, ii) steroid glycosides, and iii) steroid alkaloid glycosides.

In addition to saponins comprising triterpene saponins, the present invention also pertains to saponins comprising steroid sapogenins derived from a furostan skeleton or a spirostan skeleton. The present invention further pertains to saponins comprising steroid alkaloid sapogenins derived from a solanidan skeleton or a spirosolan skeleton. The steroid alkaloid glycosides, or glycoalkaloids, share many physical and biological properties with steroid glycosides, but alkaloid glycosides are usually considered separately because their steroidal structure contains nitrogen.

Triterpene glycosides represent one preferred class of saponins according to the present invention. The pentacyclic triterpenes can be divided into three main classes, depending on whether they have a β-amyrin, α-amyrin or lupeol skeleton.

According to the present invention, saponins in the form of triterpene glycosides preferably comprises an aglycone skeleton selected from the group of compounds consisting of Oleane (β-Amyrin), Ursane (α-Amyrin), Lupane, Taraxastane, Friedelane, Glutinane, Hopane, Dammarane, Lanostane, Holostane, and Cycloartane.

The triterpene aglycone may be hydroxylated at C-3 and certain methyl groups may be oxidized to hydroxymethyl, aldehyde or carboxyl functionalities. When an acid moiety is esterified to the triterpene aglycone, the term ester saponin is used for the respective glycosides. Further important structural elements of this class is: The unsaturation at C-12(13); the functionalization of the methyl group of C-28, C-23 or C-30; and polyhydroxylation at C-2, C-7, C-11, C-15, C-16, C-19. The formation of an additional ring structure is possible through etherification or lactonization, and esterification by aliphatic acids is also possible.

There are numerous structural variants of the triterpene glycoside class of saponins comprising a oleanane skeleton (olean-12-en skeleton). A number of such aglycone variants pertaining to the present invention are listed in Table 1 herein below.

Other representative variants of triterpene aglycones according to the present invention are listed in Table 2. The aglycones of Table 2 are representative of aglycones which do not have an olean-12-en skeleton.

A review article by Tschesche and Wulff (1972), incorporated herein by reference, gives further references and examples, together with physical constants, of both oleananes and other triterpenes. Mahato and coworkers (Das and Mahato, 1983; Mahato et al. 1992; both of which are incorporated herein by reference) have published lists of recently isolated triterpenes (not necessarily from saponins) with their. physical constants and plant sources.

Oleanane triterpenes (and some of their glycosides) have been the subject of an update (Mallavarapu, 1990; incorporated herein by reference), covering various aspects of their occurrence and chemistry. Another authoritative source of information on the triterpenes is the book written by Boiteau and colleagues (1964; incorporated herein by reference).

TABLE 1
Structures of commonly occurring olean-12-en aglycones

| No. | Olean-12-en aglycone | —OH | ═O | —COOH | Other |
|---|---|---|---|---|---|
| 1 | β-Amyrin | 3β | | | |
| 2 | Oleanolic acid | 3β | | 28 | |
| 3 | Epikatonic acid | 3β | | 29 | |
| 4 | α-Boswellic acid | 3α | | 24 | |
| 5 | Momordic acid | 3β | 1 | 28 | |
| 6 | Glycyrrhetinic acid | 3β | 11 | 30 | |
| 7 | Gypsogenin | 3β | 23 | 28 | |
| 8 | Gypsogenic acid | 3β | | 23,28 | |
| 9 | Cincholic acid | 3β | | 27,28 | |
| 10 | Serjanic acid | 3β | | 28 | 30-COOMe |
| | (30-O-methyl- | | | | |
| | spergulagenate) | | | | |
| 11 | Maniladiol | 3β,16β | | | |
| 12 | Sophoradiol | 3β,22β | | | |
| 13 | 3β,22β-Dihydroxyolean- | 3β,22β | | 29 | |
| | 12-en-29-oic acid | | | | |
| 14 | 2β-Hydroxyoleanolic | 2β,3β | | 28 | |
| | acid | | | | |
| 15 | Maslinic acid | 2α,3β | | 28 | |
| 16 | Echinocystic acid | 3β,16α | | 28 | |
| 17 | Hederagenin | 3β,23 | | 28 | |
| 18 | Phytolaccagenic acid | 3β,23 | | 28 | |
| 19 | Siaresinolic acid | 3β,19α | | 28 | |
| 20 | 21β-Hydroxyoleanolic | 3β,21β | | 28 | |
| | acid (machaerinic acid) | | | | |
| 21 | 29-Hydroxyoleanolic acid | 3β,29 | | 28 | |
| 22 | Azukisapogenol | 3β,24 | | 29 | |
| 23 | Soyasapogenol E | 3β,24 | 22 | | |
| 24 | Primulagenin D | 3β,16α | 28 | | |
| | (28-dehydroprimulagenin) | | | | |
| 25 | 3β,24-Dihydroxyolean- | 3β,24 | | | 15-en |
| | 12,15-dien-28-oic acid | | | | |
| 26 | Soyasapogenol C | 3β,24 | | | 21-en |
| 27 | Glabrinic acid | 3β,26 | 11 | 30 | |
| 28 | Quillaic acid | 3β,16 | 23 | 28 | |
| 29 | 21β-Hydroxygypsogenin | 3β,21 | 23 | 28 | |
| 30 | Barringtogenic acid | 2α,3β | | 23,28 | |
| 31 | Medicagenic acid | 2β,3β | | 23,28 | |
| 32 | Dianic acid | 3β,29 | | 23,28 | |
| 33 | Soyasapogenol B | 3β,22β,24 | | | |
| 34 | 3β,22β,24-Trihydroxyolean- | 3β,22β,24 | | 29 | |
| | 12-en-29-oic acid | | | | |
| 35 | Primulagenin A | 3β,I6α,28 | | | |
| 36 | 2β,3β,28-Trihydroxyolean- | 2β,3β,28 | | | |
| | 12-en | | | | |
| 37 | Priverogenin A | 3β,16α,22α | | 28 | |
| 38 | 16α-Hydroxyhederagenin | 3β, I6α,23 | | 28 | |
| | (caulophyllogenin) | | | | |
| 39 | 21β-Hydroxyhederagenin | 3β,21β,23 | | 28 | |
| 40 | 3β,21β,22β-Trihydroxyolean- | 3β,21β,22β | | 29 | |
| | 12-en-29-oic acid | | | | |
| 41 | 23-Hydroxyimberbic acid | 1α,3β,23 | | 29 | |
| 42 | Arjunic acid | 2α,3β,I9α | | 28 | |
| 43 | Arjunolic acid | 2α,3β,23 | | 28 | |
| 44 | Asterogenic acid | 2β,3β,I6α | | 28 | |
| 45 | Bayogenin | 2β,3β,23 | | 28 | |
| 46 | 16-Hydroxymedicagenic | 2β,3β,16 | | 23,28 | |
| | acid | | | | |
| 47 | Presenegenin | 2β,3β,27 | | 23,28 | |
| 48 | Jaligonic acid | 2β,3β,23 | | 28,30 | |
| 49 | Phytolaccagenin | 2β,3β,23 | | 28 | 30-COOMe |
| 50 | Belleric acid | 2α,3β,23,24 | | | |
| 51 | Barringtogenol A | 2α,3β,23,28 | | | |
| 52 | Protobassic acid | 2β,3β,6β, | | 28 | |
| | | 23 | | | |
| 53 | Platycogenic acid C | 2β,3β,16β, | | 28 | |
| | | 21β | | | |
| 54 | Polygalacic acid | 2β,3β,16α, | | 28 | |
| | | 23 | | | |
| 55 | Tomentosic acid | 2α,3β,19β, | | 28 | |
| | | 23 | | | |
| 56 | Arjungenin | 2α,3β,I9β, | | 28 | |
| | | 23 | | | |
| 57 | Esculentagenic acid | 2β,3β,23, | | 28 | |
| | | 30 | | | |
| 58 | 23-Hydroxylongispinogenin | 3β,16β,23, | | | |
| | | 28 | | | |
| 59 | Cyclamiretin E | 3β,16α,28, | | | |
| 60 | Soyasapogenol A | 3β,21β, | | | |
| | | 22β,24 | | | |
| 61 | Oxytrogenol | 3β,22β,24, | | | |
| | | 29 | | | |
| 62 | 3α,21β,22α,28-Tetrahydroxyolean- | 3α,21β, | | | |
| | 12-en | 22α,28 | | | |
| 63 | 3β,23,27,29-Tetrahydroxyoleanolic | 3β,23,27, | | 28 | |
| | acid | 29 | | | |
| 64 | Barringtogenol C | 3β,16α, | | | |
| | | 21β,22α, | | | |
| | | 28 | | | |
| 65 | Camelliagenin C | 3β,16α, | | | |
| | | 22α,23, | | | |
| | | 28 | | | |
| 66 | 16α-Hydroxyprotobassic | 2β,3β,6β, | | 28 | |
| | acid | 16α,23 | | | |
| 67 | Platycodigenin | 2β,3β,16α, | | 28 | |
| | | 23,24 | | | |
| 68 | Protoaescigenin | 3β,16α, | | | |
| | | 21β,22α, | | | |
| | | 24,28 | | | |
| 69 | Theasapogenol A | 3β,16α, | | | |
| | | 21β,22α, | | | |
| | | 23,28 | | | |
| 70 | R₁-Barrigenol | 3β,15α, | | | |
| | | 16α,21β, | | | |
| | | 22α,28 | | | |

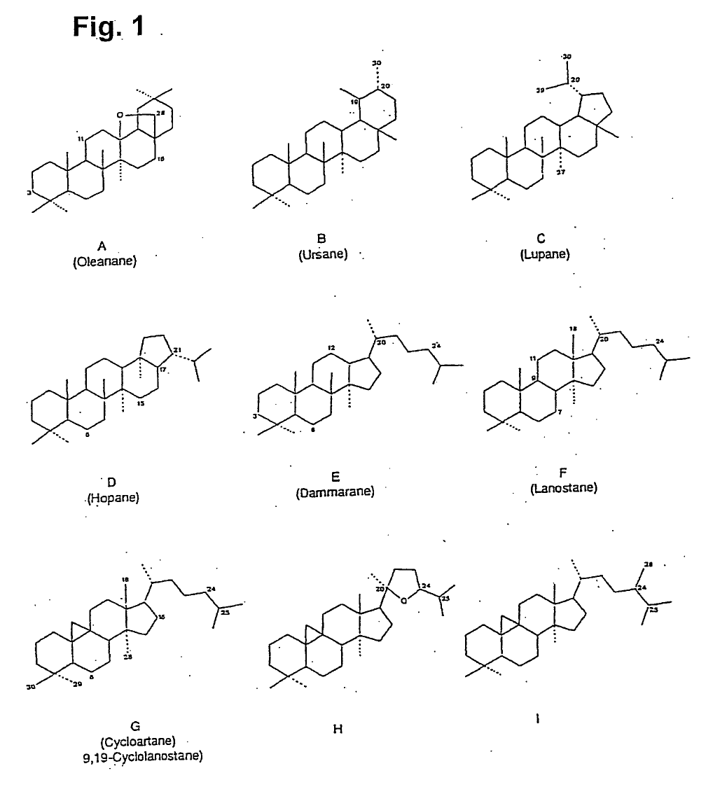

TABLE 2
Triterpene aglycones (other than olean-12-en type). The skeletons are
designated by capital letters A to I as indicated in FIG. 1.

| No. | Name | Skeleton | —OH | ═O | —COOH | Other |
|---|---|---|---|---|---|---|
| 71 | Protoprimulagenin A | A | 3β,16α | | | |
| 72 | Cyclamiretin A | A | 3β,16α | 30 | | |
| 73 | Rotundiogenin A | A | 3β,16α | | | 11-en |
| 74 | Saikogenin E | A | 3β,16α | | | 11-en |
| 75 | Anagalligenone | A | 3β,23 | 16 | | |
| 76 | Saikogenin F | A | 3β,16β,23 | | | |
| 77 | Saikogenin G | A | 3β,16α,23 | | | |
| | (anagalligenin B) | | | | | |
| 78 | Priverogenin B | A | 3β,16α,22α | | | |
| 79 | Anagalligenin A | A | 3β,16α, | | | |
| | | | 22α,28 | | | |
| 80 | α-Amyrin | B | 3β | | | 12-en |
| 81 | Ursolic acid | B | 3β | | 28 | 12-en |
| 82 | Quinovic acid | B | 3β | | 27,28 | 12-en |
| 83 | 3β-Hydroxyurs- | B | 3β | | 27,28 | 12,20(30)- |
| | 12,20(30)-dien- | | | | | dien |
| | 27,28-dioic acid | | | | | |
| 84 | Pomolic acid | B | 3β,19α | | 28 | 12-en |
| 85 | Ilexgenin B | B | 3β,19α | | 28 | 12-en, |
| | | | | | | (30S)30β |
| 86 | Ilexgenin A | B | 3β,19α | | 24,28 | 12-en |
| 87 | 21β-Hydroxyursolic | B | 3β,21β | | 28 | 12-en |
| | acid | | | | | |
| 88 | 23-Hydroxyursolic | B | 3β,23 | | 28 | 12-en |
| | acid | | | | | |
| 89 | 3β,23-Dihydroxy- | B | 3β,23 | | 28 | 20-en |
| | taraxer-20-en-28- | | | | | |
| | oic acid | | | | | |
| 90 | Rotundic acid | B | 3β,19α,23 | | 28 | 12-en |
| 91 | Rotungenic acid | B | 3β,19α,24 | | 28 | 12-en |
| 92 | Madasiatic acid | B | 2α,3β,6β | | 28 | 12-en |
| 93 | Asiatic acid | B | 2α,3β,23 | | 28 | 12-en |
| 94 | Euscaphic acid | B | 2α,3α,I9α | | 28 | 12-en |
| 95 | Tormentic acid | B | 2α,3β,19α | | 28 | 12-en |
| 96 | 2α,3β,19α-Trihydroxyurs- | B | 2α,3α,I9α | | 23,28 | 12-en |
| | 12-en- | | | | | |
| | 23,28-dioic acid | | | | | |
| 97 | 6β-Hydroxytormentic | B | 2α,3β,6β, | | 28 | 12-en |
| | acid | | 19α | | | |
| 98 | 7α-Hydroxytormentic | B | 2α,3β,7α, | | 28 | 12-en |
| | acid | | 19α | | | |
| 99 | 23-Hydroxytormentic | B | 2α,3β, | | 28 | 12-en |
| | acid | | 19α,23 | | | |
| 100 | 24-Hydroxytormentic | B | 2α,3β, | | 28 | 12-en |
| | acid | | 19α,24 | | | |
| 101 | 1α,3β,19α,23-Tetrahydroxyurs- | B | 1α,3β, | | 28 | 12-en |
| | 12-en- | | 19α,23 | | | |
| | 28-oic acid | | | | | |
| 102 | Madecassic acid | B | 2α,3β,6β, | | 28 | 12-en |
| | | | 23 | | | |
| 103 | 6β,23-Dihydroxytormentic | B | 2α,3β,6β, | | 28 | 12-en |
| | acid | | 19α,23 | | | |
| 104 | Lupeol | C | 3β | | | 20(29)-en |
| 105 | Betulin | C | 3β,28 | | | 20(29)-en |
| 106 | Betulinic acid | C | 3β | | 28 | 20(29)-en |
| 107 | 3-epi-Betulinic acid | C | 3α | | 28 | 20(29)-en |
| 108 | 3β,23-Dihydroxylup- | C | 3β,23 | | 28 | 20(29)-en |
| | 20(29)-en-oic acid | | | | | |
| 109 | 3α-Hydroxylup- | C | 3α | | 23,28 | 20(29)-en |
| | 20(29)-en-23,28- | | | | | |
| | dioic acid | | | | | |
| 110 | 3α,IIα-Dihydroxylup- | C | 3α,IIα | | 23,28 | 20(29)-en |
| | 20(29)-en-23,28- | | | | | |
| | dioic acid | | | | | |
| 111 | Cylicodiscic acid | C | 3β,27α | | 28 | 20(29)-en |
| 112 | Mollugogenol B | D | 3β,6α | | | 15,17(21)- |
| | | | | | | dien |
| 113 | (20S)-Protopanaxadiol | E | 3β,12β, | | | 24-en |
| | | | 20S | | | |
| 114 | (20S)-Protopanaxatriol | E | 3β,6α, | | | 24-en |
| | | | 12β, | | | |
| | | | 20S | | | |
| 115 | Bacogenin A₁ | E | 3β,19,20 | | 16 | 24-en |
| 116 | Seychellogenin | F | 3β | | | 7,9(11)- |
| | | | | | | dien |
| | | | | | | 18,20- |
| | | | | | | lactone |
| 117 | Mollic acid | G | 1α,3β | | 28 | |
| 118 | 3β,21,26-Trihydroxy- | G | 3β,21,26 | | | 24-en |
| | 9,19-cyclolanost- | | | | | |
| | 24-en | | | | | |
| 119 | Thalicogenin | G | 3β,16β, | | | 24-en |
| | | | 22,28 | | | |
| 120 | 3β,16β,24,25-Tetrahydroxy- | G | 3β,16β, | | | |
| | 9,19-cyclo- | | 24,25 | | | |
| | lanostane | | | | | |
| 121 | 3β,6α,16β,24,25- | G | 3β,6α, | | | |
| | Pentahydroxy-9,19- | | 16β,24, | | | |
| | cyclolanostane | | 25 | | | |
| 122 | Cycloastragenol | H | 3β,6α, | | | |
| | (astramembrangenin, | | 16β,25 | | | |
| | cyclosiversigenin) | | | | | |
| 123 | 3β-Hydroxy-9,19- | I | 3β | | | 24(28)-en |
| | cyclolanost-24(28)- | | | | | |
| | en | | | | | |
| 124 | Jessic acid | I | 1α,3β | 23 | 29 | 24(28)-en |

The posintros according to the present invention have molecular weights ranging from for example about 400 daltons to more than 2,000 daltons. Further examples are from about 500 daltons, such as from about 600 daltons, for example from about 700 daltons, such as from about 800 daltons, such as from about 900 daltons, for example from about 1000 daltons, such as from about 1100 daltons, such as from about 1200 daltons, for example from about 1300 daltons, such as from about 1400 daltons, such as from about 1500 daltons, for example from about 1600 daltons, such as from about 1700 daltons, such as from about 1800 daltons, for example from about 1900 daltons, such as from about 2000 daltons, to preferably less than 4,000 daltons.

In one embodiment of the present invention, the saponin compound is acylated with one or more organic acids such as acetic acid, malonic acid, angelic acid and the like (see fx Massiot, G. & Lavaud, C., Stud. Nat. Prod. Chem. 15:187-224 (1995), incorporated herein by reference).

Saponins according to the present invention, including the aglycone as illustrated in Table 1 and Table 2 herein above, have one or more linear or branched saccharide chains attached to the aglycone part via a glycosidic ether or ester bond.

According to the number of saccharide chains attached to the aglycone, the saponins can be monodesmosidic saponins (with a single saccharide chain), or bidesmosidic saponins (with two saccharide chains).

In the monodesmosidic saponins according to the invention, the saccharide chain is preferably attached by a glycosidic ether linkage at the C-3 of the aglycone. In addition to the C-3 linked saccharide chain, bidesmosidic saponins have a second saccharide chain bound at C-28 (triterpene saponins) or at C-26 (steroid saponins) by an ester linkage. Because of the typical lability of esters, bidesmosidic saponins are readily converted into their monodesmosidic forms by mild hydrolysis (Hostettmann, K., et al., Methods Plant Biochem. 7:435-471 (1991)).

Bidesmosidic saponins according to the invention preferably have two sugar chains, one of which may be attached through an ether linkage at C-3, and one attached linkage (acyl glycoside) at C-28 (triterpene saponins), or an ether linkage at C-26 (furostanol saponins).

Bidesmosidic saponins are easily converted into monodesmosidic saponins by, for example, hydrolysis of the esterified sugar at C-28 in triterpene saponins, and they differ from monodesmosidic saponins with respect to some properties and activities. Also, when one sugar chain is attached at C-3, a second sugar group may be esterified to the carboxyl group at C-17 of the aglycone. Furthermore, some dammarane glycosides and lanostane glycosides may have a second or even a third glycosidically bound sugar chain.

Tridesmosidic saponins according to the invention have three sugar chains. Hydrolysis of the esterified sugars result in conversion into bidesmosidic saponins and/or monodesmosidic saponins. An example of one tridesmosidic triterpene is a 9,19-cyclolanostane (cycloartane) substituted glycosidically at positions C-3, C-6 and C-25. An example of a tridesmosidic olean-12-en saponin is quinoside A, in which sugars are attached at positions C-3, C-23 and C-28 of hederagenin (Meyer et al. 1990). Also, a tridesmoside of 16α-hydroxymedicagenic acid (zahnic acid) has been found in the aerial parts of alfalfa (Medicago saliva, Leguminosae) (Oleszek et al. 1992).

The saccharide moiety of saponins according to the invention may be linear or branched, with about 11 being the highest number of monosaccharide units yet found in a saponin (Clematoside C from Clematis manshurica (Ranunculaceae); Khorlin et al. 1965). However, the present invention is not limited to saccharide moieties containing 11 or less monosaccharide units. Saponins according to the present invention may comprise less than 10 saccharide moieties, such as less than 9 saccharide moieties, for example less than 8 saccharide moieties, such as less than 7 saccharide moieties, for example less than 6 saccharide moieties, such as less than 5 saccharide moieties, for example less than 4 saccharide moieties, such as less than 3 saccharide moieties, for example less than 2 saccharide moieties, such as one saccharide moiety.

In fact, most saponins so far isolated tend to have relatively short (and often unbranched) sugar chains, containing from about 2 to about 5 monosaccharide residues. Kochetkov and Khorlin (1966) have introduced the term oligoside for glycosides containing more than 3 to 4 monosaccharides.

Accordingly, there are also provided saponins according to the present invention comprising one or more sugar chains, for example two or three sugar chains, wherein one or more sugar chains comprises for example from about 2 to about 11 monosaccharide residues, such as from about 2 to about 10 monosaccharides, for example from about 2 to about 11 monosaccharide residues, such as from about 2 to about 10 monosaccharides, for example from about 2 to about 9 monosaccharide residues, such as from about 2 to about 8 monosaccharides, for example from about 2 to about 7 monosaccharide residues, such as from about 2 to about 6 monosaccharides, for example from about 2 to about 5 monosaccharide residues, such as from about 2 to about 4 monosaccharides, for example from about 2 to about 3 monosaccharide residues.

In another embodiment the present invention provides saponins comprising one or more sugar chains, for example two or three sugar chains, wherein one or more sugar chains comprises more than 3 monosaccharides, such as more than 4 monosaccharides, for example more than 5 monosaccharides, such as more than 6 monosaccharides, for example more than 7 monosaccharides, such as more than 8 monosaccharides, for example more than 9 monosaccharides, and preferably less than 11 monosaccharide residues.

Oligosides as used herein refer to saponin glycosides containing 4 or more monosaccharides, such as more than 5 monosaccharides, for example more than 6 monosaccharides, and independently thereof preferably less than 12 monosaccharides, such as less than 11 monosaccharides, for example less than 10 monosaccharides, such as less than 9 monosaccharides, for example less than 8 monosaccharides.

However, saponins according to the present invention may also comprise one or more sugar chains, for example two or three sugar chains, wherein one or more of said sugar chains comprises more than 11 monosaccharides, for example about 15 monosaccharides, such as about 20 monosaccharides, for example more than about 25 monosaccharides, such as more than about 40 monosaccharides.

Preferred monosaccharide residues of saponin glycosides according to the present invention are: D-glucose (Glc), D-galactose (Gal), D-glucuronic acid (GlcA), D-galacturonic acid (GalA), L-rhamnose (Rha), L-arabinose (Ara), D-xylose (Xyl) and D-fucose (Fuc). Also preferred are D-apiose (Api), D-ribose (Rib), and D-allose (All). Furthermore, saponins obtained from marine organisms often contain D-quinovose (Qui) (sometimes written as D-chinovose). All the above abbreviations are used in accordance with IUPAC recommendations (Pure Appl. Chem. (1982), vol. 54, p. 1517-1522).

In addition to the above-mentioned monosaccharides the present invention also pertains to unusual monosaccharides such as uronic acids that are known to occur in some triterpene glycosides. Another example of unusual monosaccharides are monosaccharides comprising an amino saccharide and/or an acylated saccharide.

Among the preferred monosaccharides directly attached to the saponin aglycone are glucose, arabinose, glucuronic acid and xylose. Such monosaccharides thus forms the link between the saccharide part and the aglycone part of the saponin.

Another group of saccharides according to the invention are saccharides comprising acylated sugar moieties, as well as saccharides comprising methylated and/or sulphated sugar moieties.

In accordance with generally agreed nomenclature, the configurations of the interglycosidic linkages are given herein by α and β, respectively, and the monosaccharides making up the sugar part of saponins according to the invention may adopt a pyranose (p) and/or a furanose (f) form.

As used herein, saponins from the bark of the Quillaja saponaria Molina tree are termed Quillaja saponins. Quillaja saponins represent one group of particularly preferred saponins according to the present invention. Quillaja saponins are either first saponins or second saponins, wherein the latter group of Quillaja saponins are capable of forming and interaction with a genetic determinant.

Quillaja saponins are found as admixture of about twenty structurally closely related triterpenoid glycosides with minimal differences between them (Higuchi, R. et al., Phytochemistry 26:229 (1987); ibid., 26:2357 (1987); ibid., 27:1169 (1988); Kensil et al., U.S. Pat. No. 5,057,540 (1991); Kensil et al., Vaccines 92:35 (1992)). Quillaja saponins are chemically and immunologically well-characterized (see fx Dalsgaard, K. Arch. Gesamte Virusforsch. 44:243 (1974); Dalsgaard, K., Acta Vet. Scand. 19 (Suppl. 69):1 (1978); Higuchi, R. et al., Phytochemistry 26:229 (1987); ibid. 26:2357 (1987); ibid. 27:1168 (1988); Kensil, C. et al., J. Immunol. 146:431 (1991); Kensil et al., U.S. Pat. No. 5,057,540 (1991); Kensil et al., Vaccines 92:35 (1992); Bomford, R. et al., Vaccine 10:572 (1992); Kensil, C. et al., U.S. Pat. No. 5,273,965 (1993);); Kensil, C. et al., U.S. Pat. No. 5,443,829 (1995);); Kensil, C. et al., U.S. Pat. No. 5,583,112 (1996); and Kensil, C. et al., U.S. Pat. No. 5,650,398 (1997); all of which are incorporated herein by reference).

Quillaja saponins belong to a family of closely related O-acylated triterpene glycoside structures. They have an aglycone triterpene (quillaic acid), with branched saccharide chains attached to positions 3 and 23, and an aldehyde group in position 23. A unique characteristic of Quillaja saponins pertaining to the invention is the presence of certain acyloil acyl moieties linked at the C-3 hydroxy group of a fucopyranose bound by an ester bond to position 28 of quillaic acid. Preferred acyl moieties are 3,5-dihydroxy-6-methyloctanoic acid, 3,5-dihydroxy-6-methyloctanoic acid, 5-O-α-L-rhamno-pyranosyl-(1→2)-α-L-arabino-furanoside, and 5O-α-L-arabino-furanoside.

Quillaja saponins according to the present invention may be obtained from quillaja plant species including Quillaja saponaria Molina and others either as a crude extract, or as an extract which have been purified by various open column techniques (i.e. chromatography by means of e.g. ion exchange-, size exclusion-, hydrophobic-, affinity-, and otherwise). Such purified or semi-purified saponins are generally referred to in the art as “Quil A”, or “Quadri A”, as described by WO 95/09179, which is incorporated herein by reference.

The saponins may also be purified by high resolution hydrophobic interaction techniques, such as e.g. HPLC, and this form of purification generates fractions known in the art as e.g. “Quadri 1”, “Quadri2”, and the like (see e.g. WO 95/09179, as well as Kamstrup et al. Vaccine (2000), vol 18, no. 21, 2244-2249, incorporated herein by reference).

Particularly preferred are saponin extracts from Quillaja saponaria Molina, primarily the DQ-extract produced according to K. Dalsgaard: Saponin Adjuvants, Bull. Off. Int. Epiz. 77 (7-8), 1289-1295 (1972), and Quil A which is produced according to K. Dalsgaard: Saponin Adjuvants III, Archiv fur die Gesamte Virusforschung 44, 243-254 (1974). Also mixtures of such glycosides pertain to the present invention.

The amount of glycoside added should be at least 1-3 times their critical micelle formation concentration (CMC), preferably at least 5, especially at least 7-12 times. Preferably Quil A is used, which has a critical micelle formation concentration of 0.03% by weight. The amount of Quil A should then be at least 0.02% by weight, especially 0.05-0.5% by weight, preferably 0.2% by weight.

Further fractions of saponins according to the present invention are described in detail herein below. According to U.S. Pat. No. 5,057,540, the contents of which are incorporated herein by reference, saponins can be purified from an aqueous extract of the bark of the South American tree, Quillaja saponaria Molina. At least 22 peaks with saponin activity were separable.

The predominant purified Quillaja saponins are QA-7, QA-17, QA-18, and QA-21. These saponins have been purified by high pressure liquid chromatography (HPLC) and low pressure silica chromatography. QA-21 can be further purified using hydrophilic interaction chromatography (HPLC) and resolved into two peaks, QA-21-V1 and QA-21-V2, that are different compounds.

Thus, “QA-21” designates the mixture of components QA-21-V1 and QA-21-V2 that appear as a single peak on reversed-phase HPLC on VYDAC C4 (5 μm particle size, 330 Ångstrøm pore, 4.6 mm ID×25 cm L) in 40 mM acetic acid in methanol/water (58/42, v/v). The component fractions are referred to specifically as QA-21-V1 and QA-21-V2 when describing experiments or results performed on the further purified components.

In order to purify saponins from Quillaja saponaria Molina bark, aqueous extracts of the Quillaja saponaria Molina bark are dialyzed against water. The dialyzed extract is lyophilized to dryness, extracted with methanol and the methanol-soluble extract is further fractionated by silica gel chromatography and by reversed-phase high pressure liquid chromatography (RP-HPLC).

TABLE 3
Reversed-phase HPLC peaks designating individual
saponins and their corresponding retention times.

| Peak | Retention Time (minutes) |
|---|---|
| QA-1 | solvent front |
| QA-2 | 4.6 |
| QA-3 | 5.6 |
| QA-4 | 6.4 |
| QA-5 | 7.2 |
| QA-6 | 9.2 |
| QA-7 | 9.6 |
| QA-8 | 10.6 |
| QA-9 | 13.0 |
| QA-10 | 17.2 |
| QA-11 | 19.0 |
| QA-12 | 21.2 |
| QA-13 | 22.6 |
| QA-14 | 24.0 |
| QA-15 | 25.6 |
| QA-16 | 28.6 |
| QA-17 | 35.2 |
| QA-18 | 38.2 |
| QA-19 | 43.6 |
| QA-20 | 47.6 |
| QA-21 | 51.6 |
| QA-22 | 61.0 |

As shown above, individual saponins can be separated by reversed-phase HPLC. At least 22 peaks (designated QA-1 to QA-22) are separable. Individual components are identified by retention time on a VYDAC C4 HPLC column as follows in Table 3 herein above. Each peak corresponds to a carbohydrate peak that exhibits only a single band on reversed-phase thin layer chromatography.

The substantially pure QA-7 saponin is characterized as having immune adjuvant activity, containing about 35% carbohydrate (as assayed by anthrone) per dry weight, having a UV absorption maximum of 205-210 nm, a retention time of approximately 9-10 minutes on RP-HPLC on a VYDAC C4 column having 5 μm particle size, 330 Ångstrøm pore, 4.6 mm ID×25 cm L in a solvent of 40 mM acetic acid in methanol-water (58/42; v/v) at a flow rate of 1 mL/min, eluting with 52-53% methanol from a VYDAC C4 column having 5 μm particle size, 330 Ångstrøm pore, 10 mm ID×25 cm L in a solvent of 40 mM acetic acid with. gradient elution from 50 to 80% methanol, having a critical micellar concentration of 0.06% (w/v) in water and 0.07% (w/v) in phosphate buffered saline, causing no detectable hemolysis of sheep red blood cells at concentrations of 200 μg/mL or less, and containing the monosaccharide residues terminal rhamnose, terminal xylose, terminal glucose, terminal galactose, 3-xylose, 3,4-rhamnose, 2,3-fucose, 2,3-glucuronic acid, and apiose (linkage not determined).

The substantially pure QA-17 saponin is characterized as having adjuvant activity, containing about 29% carbohydrate (as assayed by anthrone) per dry weight, having a UV absorption maximum of 205-210 nm, a retention time of approximately 35 minutes on RP-HPLC on a VYDAC C4 column having 5 μm particle size, 330 Ångstrøm pore, 4.6 mm ID×25 cm L in a solvent of 40 mM acetic acid in methanol-water (58142; v/v) at a flow rate of 1 mL/min, eluting with 63-64% methanol from a VYDAC C4 column having 5 μm particle size, 330 Ångstrøm pore, 10 mm ID×25 cm L in a solvent of 40 mM acetic acid with gradient elution from 50 to 80% methanol, having a critical micellar concentration of 0.06% (w/v) in water and 0.03% (w/v) in phosphate buffered saline, causing hemolysis of sheep red blood cells at 25 μg/mL or greater, and containing the monosaccharide residues terminal rhamnose, terminal xylose, 2-fucose, 3-xylose, 3,4-rhamnose, 2,3-glucuronic acid, terminal glucose, 2-arabinose, terminal galactose and apiose (linkage not determined).

The substantially pure QA-18 saponin is characterized as having immune adjuvant activity, containing about 25-26% carbohydrate (as assayed by anthrone) per dry weight, having a UV absorption maximum of 205-210 nm, a retention time of approximately 38 minutes on RP-HPLC on a VYDAC C4 column having 5 μm particle size, 330 Ångstrøm pore, 4.6 mm ID×25 cm L in a solvent of 40 mM acetic acid in methanol/water (58/42; v/v) at a flow rate of 1 mL/min, eluting with 64-65% methanol from a VYDAC C4 column having 5 μm particle size, 330 Ångstrøm pore, 10 mm ID×25 cm L in a solvent of 40 mM acetic acid with gradient elution from 50 to 80% methanol, having a critical micellar concentration of 0.04% (w/v) in water and 0.02% (w/v) in phosphate buffered saline, causing hemolysis of sheep red blood cells at concentrations of 25 μg/mL or greater, and containing the monosaccharides terminal arabinose, terminal apiose, terminal xylose, terminal glucose, terminal galactose, 2-fucose, 3-xylose, 3,4-rhamnose, and 2,3-glucuronic acid.

The substantially pure QA-21 saponin is characterized as having immune adjuvant activity, containing about 22% carbohydrate (as assayed by anthrone) per dry weight, having a UV absorption maximum of 205-210 nm, a retention time of approximately 51 minutes on RP-HPLC on a VYDAC C4 column having 5 μm particle size, 330 Ångstrøm pore, 4.6 mm ID×25 cm L in a solvent of 40 mM acetic acid in methanol/water (58/42; v/v) at a flow rate of 1 mL/min, eluting with 69 to 70% methanol from a VYDAC C4 column having 5 μm particle size, 330 Ångstrøm pore, 10 mm ID×25 cm L in a solvent of 40 mM acetic acid with gradient elution from 50 to 80% methanol, with a critical micellar concentration of about 0.03% (w/v) in water and 0.02% (w/v) in phosphate buffered saline, and causing hemolysis of sheep red blood cells at concentrations of 25 μg/mL or greater. The component fractions, substantially pure QA-21-V1 and QA-21-V2 saponins, have the same molecular weight and identical spectra by fast atom bombardment-mass spectroscopy (FAB-MS). They differ only in that QA-21-V1 has a terminal apiose that is xylose in QA-21-V2 (which therefore has two terminal xyloses and no apiose). The two components additionally contain the monosaccharides terminal arabinose, terminal apiose, terminal xylose, 4-rhamnose, terminal galactose, 2-fucose, 3-xylose, and 2,3-glucuronic acid.

The alkaline hydrolysis products can be prepared as follows. Treatment of QA-18 by brief alkaline hydrolysis yielded one major carbohydrate-containing alkaline hydrolysis product (designated QA-18-H). Purified QA-18-H was prepared from QA-18 and isolated in the following manner:

One mL QA-18 (5 mg/ml) was incubated with 25 μl 1N NaOH for 15 minutes at room temperature. The reaction was stopped with the addition of 100 μl 1N acetic acid. Using these hydrolysis conditions, QA-18 was completely converted to a major hydrolysis product (QA-18-H) eluting in a peak with retention time of 8.0 min compared to 66.8 min for unhydrolyzed QA-18, indicating the increased hydrophilicity of QA-18-H. (Chromatography on VYDAC C4 (4.6 mm ID×25 cm L) in 0.1% trifluoroacetic acid in 55/45 methanol/water (v/v) and eluted in a gradient to 64/36 methanol/water (v/v) over 180 minutes, flow rate of 1 mL/minute). The peak containing pure QA-18-H (retention time 8.0 min) was pooled for further characterization. The hydrolysis product of QA-21, designated QA-21-H, was prepared and purified in the same manner. QA-21-H had a retention time of 9.3 minutes compared to 80.4 minutes for its unhydrolyzed precursor, QA-21. The hydrolysis products were shown by retention time on HPLC and by reversed-phase thin layer chromatography to be identical to major hydrolysis products generated using the method of Higuchi et al., Phytochemistry 26:229 (1987) using mild alkaline hydrolysis in NH₄HCO₃ (Table 4).

TABLE 4
Retention Time of Major Alkaline Hydrolysis Products

| | |
|---|---|
| QA-17-H | 8.0^{a} |
| QA-18-H | 8.0^{a} |
| | 8.2^{b} |
| QA-21-H | 9.3^{a} |
| | 9.5^{b} |
| Hydrolyzed - “Quil-A” | 8.2^{a}, 9.3^{a} |

^{a}Cambridge Biotech hydrolysis conditions: 5 mg/ml saponin, pH 13, reaction time = 15 minutes at room temperatur.
^{b}Higuchi et al. hydrolysis conditions: 5 mg/ml saponin, 6% NH₄HCO₃, methanol/H₂O (1/1, v/v), reaction time = 60 minutes at 100° C.

HPLC Conditions:

VYDAC C4, 5 mm particle size, 330 Ångstrøm pore size, 0.46×25 cm

Solvent A=0.1% trifluoroacetic acid in water

Solvent B—0.1% trifluoroacetic acid in methanol

Gradient=55-64% B/180 minutes

Flow rate—1 ml/min

In addition, these products, QA-18-H and QA-21-H, were shown to be the major breakdown products from hydrolysis of “Quil-A”, a crude saponin mixture containing QA-7, QA-17, QA-18, and QA-21 as well as other saponins, indicating that the hydrolysis products QA-21-H and QA-18H are the same hydrolysis products isolated by Higuchi et al., supra, for structural characterization.

Even further preferred saponins according to the present invention are those described e.g. in EP 0 436 620 B1, incorporated herein by reference, including fractions termed QHA, QHB, QHC, or similar compositions of Quillaja saponins.

Acylated quillaja saponins appear to be exceptional since their monodesmosidic forms are significantly less effective hemolytic agents than their acylated and non-acylated bidesmosidic forms (Pillion, D. J., et al., J. Pharm. Sci., 84:1276-1279 (1996)).

In addition to Quillaja saponins, saponins originating from Gypsophila species and Saponaria species, including Saponaria officinalis, are also useful in accordance with the present invention, particularly Gypsophila species and Saponaria species which have been shown to include “quillajic acid” as the aglycon component of the saponin glycoside. Furthermore, such saponins from Gypsophila species and Saponaria species preferably comprise triterpene aglycones with an aldehyde group linked or attached to position 4, branched oligosaccharides linked by an ester bond in position 28, and a 3-O-glucuronic acid (3-O-glcA) that in Quillaja and Gypsophila is linked to branched oligosaccharides. Saponins from Q. saponaria and S. jenisseenis include acyl moieties, whereas saponin from Gypsophila, Saponaria, and Acanthophyllum do not include acyl moieties. Each of these non-acylated or deacylated saponins is useful in the present invention.

Further desirable triterpene saponins are the bidesmosidic saponin, squarroside A, isolated from Acanthophyllum squarrosum; the saponin lucyoside P; and two acylated saponins isolated from Silene jenisseensis Willd. Following is a brief description of these compounds.

Squarroside A is abidesmosidic saponin that contains two oligosaccharide chains linked to C-3 and C-28 of its aglycone gypsogenin. Similar to the gypsophila saponin, it has an aldehyde group linked to C-4 of the aglycone, and a glucuronic acid residue at C-3. In addition, it contains an acetylated fucose residue at C-28. It has been shown that squarroside A has immunomodulating activity as measured by an in vitro lymphoproliferative test. These apparently nonspecific immunomodulating effects were dose-dependent: a suppressive effect at concentrations in the μg range and a stimulant effect in the pg range.

Lucyoside P is a bidesmosidic saponin that has carbohydrate residue linked to C-3 and C-28 of its aglycone quillaic acid, and an aldehyde group at C4. Lucyoside P has a glucuronic acid residue at C-3.

Two acylated saponins have been isolated from the Caryophyllacea Silene jenis-seensis. These saponins have carbohydrates linked to C-3 and C-28 of their agylcone quillaic acid. The carbohydrate residues linked to C-3 and C-28 are glucuronic acid and fucose, respectively. The fucose residue is acylated with a p-methoxy-cinnamoyl group to yield trans- and cis-p-methoxycinnamoyl tritepene glycosides.

Although the saponins mentioned herein immediately above have an aldehyde group, they apparently have no immunostimulating activity or a significantly reduced immunostimulating activity, as detected by an in vitro chemiluminescence granulocyte assay.

Yet further examples of useful saponins according to the present invention pertain to triterpensaponins such as the polar acidic bisdesmosides extracted from e.g. Chikosetsusaponin IV, Calendula-Glycoside C, Chikusetsusaponin V, Achyranthes-Saponin B, Calendula-Glycoside A, Araloside B, Araloside C, Putranjia-Saponin III, Bersamasaponoside, Putranjia-Saponin IV, Trichoside A, Trichoside B, Saponaside A, Trichoside C, Gypsoside, Nutanoside, Dianthoside C, Saponaside D, preferably aescine from Aesculus hippocastanum (T. Patt and W. Winkler: Das therapeutisch wirksame Prinzip der Rosskatanie (Aesculus hippocastanum), Arzneimittelforschung 10(4), 273-275 (1960) or sapoalbin from Gypsophilla struthium (R. Vochten, P. Joos and R. Ruyssen: Physicochemical properties of sapoalbin and their relation to the foam stability, J. Pharm. Belg. 42, 213-226 (1968).

A number of so-called “modified saponins” obtained from Quillaja saponaria have been disclosed by Kensil et al. in e.g. U.S. Pat. No. 5,273,965; U.S. Pat. No. 5,443,829; and U.S. Pat. No. 5,650398, all of which are incorporated herein by reference. The modified Quillaia saponins typically comprise a methylenealcohol group or a methyleneamino group instead of the naturally occurring triterpene aldehyde group. The modified saponins may be further modified with respect to their saccharide moieties.

One interesting saponin composition according to the present invention is the so-called “7-0-3” composition comprising 7/10 (70%) QH-A, 0/10 (0%) QH-B, and 3/10 (30%) QH-C, respectively of each fraction, as described by Ronnberg et al. in Vaccine (1995), vol. 13, no. 14, p. 1375-1382, and in Vaccine (1997), vol. 15, no. 17-18, p. 1820-1826.

The ratio between the first saponin and the second saponin in complexes in which both are present are preferably from less than 1000:1 to preferably more than 1:1000. Preferred ratios are about 100:1, for example about 80:1, such as about 60:1, for example about 50:1, such as about 40:1, for example about 30:1, such as about 25:1, for example about 20:1, such as about 18:1, for example about 16:1, such as about 14:1, for example about 12:1, such as about 10:1, for example about 9:1, such as about 8:1, for example about 7:1, such as about 6:1, for example about 5:1, such as about 4:1, for example about 3:1, such as about 2:1, for example about 1.9:1, such as about 1.8:1, for example about 1.7:1, such as about 1.6:1, for example about 1.5:1, such as about 1.4:1, for example about 1.3:1, such as about 1.2:1, for example about 1.1:1, such as about 1:1, for example about 1:1.1, such as about 1:1.2, for example about 1:1.3, such as about 1:1.4, for example about 1:1.5, such as about 1:2, for example about 1:3, such as about 1:4, for example about 1:5, such as about 1:10, for example about 1:20, such as about 1:40, for example about 1:60, such as about 1:80, for example about 1:100.

Useful sterols are in this context those who bind to saponins forming part of the posintros according to the invention. Preferred sterols are cholesterols and precursors and derivatives of thereof, as for example, phytosterols, e.g. lanosterol, lumisterol, stigmasterol, sitosterol, mycosterols, ergosterol, and thiocholesterol, the last of which can be used for binding a medicament by means of the thiol moiety. Nor-dihydro-epi-andosterol is a further preferred sterol according to the invention.

Apart from sterols, the present invention also pertains to complexes wherein at least one first and/or second sterol is substituted partly or wholly by a steroid. In one embodiment, the complexes according to the invention comprise a steroid compound instead of a sterol compound.

Steroids according to the invention are exemplified herein below in more detail. As the sterols according to the present invention comprise the characteristic skeleton structure of a steroid, the description of steroids is also a description of the skeleton of the sterols according to the present invention, one of which is cholesterol having CAS (Chemical Abstract) accession no. 57-88-5, or cationic derivatives thereof, in particular cationic derivatives obtained by linking a cationic moiety or cationic reactive group to an OH-group, including an OH-group located at position 3 of the steroid skeleton, including the OH-group of cholesterol located at position 3 (C3, or 3-OH).

All steroids are related to a characteristic molecular structure composed of 17 carbon atoms arranged in four rings conventionally denoted by the letters A, B, C, and D and bonded to 28 hydrogen atoms.

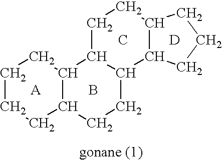

This parent structure (1), named gonane and often referred to as the steroid nucleus, may be modified in a practically unlimited number of ways by removal, replacement, or addition of a few atoms at a time; hundreds of steroids have been isolated from plants and animals, and thousands more have been prepared by chemical treatment of natural steroids or by synthesis from simpler compounds.

The steroid nucleus is a three-dimensional structure, and atoms or groups are attached to it by spatially directed bonds. Although many stereoisomers of this nucleus are possible (and may be synthesized), the saturated nuclear structures of most classes of natural steroids are alike, except at the junction of rings A and B. Simplified three-dimensional diagrams may be used to illustrate stereochemical details. For example, androstane common to a number of natural and synthetic steroids, exists in two forms (2 and 3), in which the A/B ring fusions are called cis and trans, respectively.

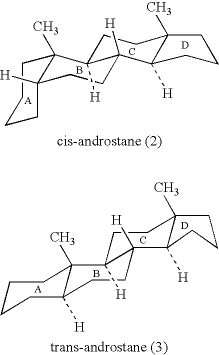

In the cis isomer, bonds to the methyl group, CH₃, and to the hydrogen atom, H, both project upward from the general plane defined by the rest of the molecule, whereas in the trans isomer the methyl group projects up and the hydrogen projects down. Usually, however, steroid structures are represented as plane projection diagrams such as 4 and 5, which correspond to 2 and 3, respectively.

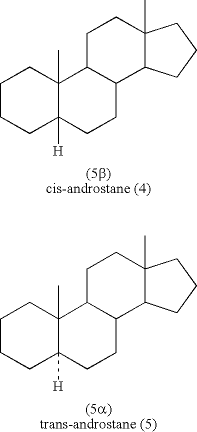

The stereochemistry of rings A and B must be specified by showing the orientation of the hydrogen atom attached at C5 (that is, carbon atom number 5; steroid numbering is explained below) as either above the plane of the diagram (designated β) or below it (α). The α-, β-symbolism is used in a similar manner to indicate the orientation of any substituent group that is attached to a saturated (fully substituted) carbon within the steroid ring system. Groups attached to unsaturated carbons lie in the same plane as the adjacent carbons of the ring system (as in ethylene), and no orientation need be specified. When the orientation of a substituent is unknown, it is assigned the symbol ξ. Bonding of β-attached substituents is shown diagrammatically as in 4 by a full line, that of α-substituents by a broken line, as in 5, and that of ξ-substituents by a wavy line.

Each carbon atom of a steroid molecule is numbered, and the number is reserved to a particular position in the hypothetical parent skeletal structure (6) whether this position is occupied by a carbon atom or not.

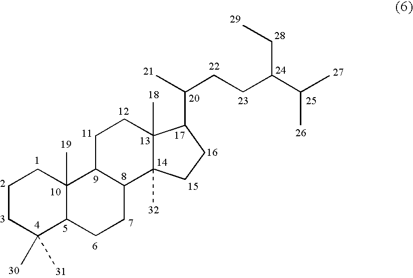

Steroids are named by modification of the names of skeletal root structures according to systematic rules agreed upon by the International Union of Pure and Applied Chemistry. By attaching prefixes and suffixes to the name of the appropriate root structure, the character of substituent groups or other structural modification is indicated. The prefixes and suffixes include numbers, called locants, indicative of the position in the carbon skeleton at which the modification occurs, and, where necessary, the orientation of a substituent is shown as α- or β-. The carbon atom at position 3, for example, is referred to as C3; a hydroxyl group attached to C3 is referred to as a 3-OH group or, more specifically, as a 3α-OH or 3β-OH group. In addition to differences in details of the steroid nucleus, the various classes of steroids are distinguished by variations in the size and structure of an atomic group (the side chain) attached at position 17. The derivations of the names of the more common root structures from those of naturally occurring compounds or classes of compounds for which they are most typical are known to the skilled artisan. For unambiguous use of such names, the orientation (α or β) of hydrogen at C5 must be specified. If no other modification is indicated, the nucleus is assumed to be as shown in (2) and (3), except in the cardanolides and bufanolides: compounds of these types characteristically possess the 5β,14β configurations, which, however, are specified.

Preferred second sterols are cationic sterols and sterols comprising at least one positively charged group at pH=7.0. Preferred sterols comprise or essentially consist of 3β-[N-(Dimethylaminoethane)-carbamoyl]cholesterol (DC-cholesterol) and/or N-(trimethylammonioethane)-carbamoylcholesterol (TC-cholesterol).

It will be understood that the second sterols comprise cationic sterols, including cationic cholesterols, wherein the OH-group (located at position 3 in cholesterol) is substituted for a positively charged group, or a group comprising at least one positive charge at pH=7.0.

The ratio between the first sterol and the second sterol in complexes in which both are present are preferably from less than 1000:1 to preferably more than 1:1000. Preferred ratios are about 100:1, for example about 80:1, such as about 60:1, for example about 50:1, such as about 40:1, for example about 30:1, such as about 25:1, for example about 20:1, such as about 18:1, for example about 16:1, such as about 14:1, for example about 12:1, such as about 10:1, for example about 9:1, such as about 8:1, for example about 7:1, such as about 6:1, for example about 5:1, such as about 4:1, for example about 3:1, such as about 2:1, for example about 1.9:1, such as about 1.8:1, for example about 1.7:1, such as about 1.6:1, for example about 1.5:1, such as about 1.4:1, for example about 1.3:1, such as about 1.2:1, for example about 1.1:1, such as about 1:1, for example about 1:1.1, such as about 1:1.2, for example about 1:1.3, such as about 1:1.4, for example about 1:1.5, such as about 1:2, for example about 1:3, such as about 1:4, for example about 1:5, such as about 1:10, for example about 1:20, such as about 1:40, for example about 1:60, such as about 1:80, for example about 1:100.

The deacylsaponins and non-acylsaponins may be directly linked to a lipophilic moiety or may be linked via a linking group. By the term “linking group” is intended one or more bifunctional molecules that can be used to covalently couple the desacylsaponins, non-acylated saponins or mixtures thereof to the lipophilic molecule.

The linker group in one embodiment covalently attaches to the carboxylic acid group of the 3-O-glucuronic acid moiety on the triterpene core structure, and to a suitable functional group present on a lipophilic moiety.

The saponins of the present invention may be directly linked to a lipophilic moiety, or a bioactive agent, including a genetic determinant, or they may be linked via a linking group. By the term “linker group” is intended one or more bifunctional molecules which can be used to covalently couple the saponin or saponin mixture to the bioactive agent including a genetic determinant. The linker group may be attached to any part of the saponin.

Typically, the saponins are linked to the lipophilic moiety, or the bioactive agent including a genetic determinant by the preparation of an active ester of glucuronic acid, a component of the saponins, followed by reaction of the active ester with a nucleophilic functional group on the bioactive agent including a genetic determinant.

Several lipophile-containing compounds, such as aliphatic amines and alcohols, fatty acids, polyethylene glycols and terpenes, can be added e.g. to the 3-O-glcA residue (3-glucuronic acid residue) of deacylsaponins, and to the 3-O-glcA residue of non-acylated saponins. The lipophile may be an aliphatic or cyclic structure that can be saturated or unsaturated. By way of example, fatty acids, terpenoids, aliphatic amines, aliphatic alcohols, aliphatic mercaptans, glycosyl-fatty acids, glycolipids, phospholipids and mono- and di-acylglycerols can be covalently attached to nonacylated saponins or desacylsaponins.

Attachment can be via a functional group on a lipophilic moiety that covalently reacts with either the acid moiety of the 3-glucuronic acid moiety, or an activated acid functionality at this position. Alternatively, a bifunctional linker can be employed to conjugate the lipophile to the 3-O-glcA residue of the first and/or second saponin.

Useful fatty acids include C₆-C₂₄ fatty acids, for example C₇-C₂₀ fatty acids, such as C₇-C₁₈ fatty acids. Examples of useful fatty acids include saturated fatty acids such as lauric, myrstic, palmitic, stearic, arachidic, behenic, and lignoceric acids; and unsaturated fatty acids, such as palmitoleic, oleic, linoleic, linolenic and arachidonic acids.

Useful aliphatic amines, aliphatic alcohols and aliphatic mercaptans include amines and alcohols and mercaptans (R—SH) having a straight-chained or branched, saturated or unsaturated aliphatic group having about 6 to about 24 carbon atoms, for example 6 to 20 carbon atoms, such as 6 to 16 carbon atoms, for example 8 to 12 carbon atoms. Examples of useful aliphatic amines include octylamine, nonylamine, decylamine, dodecylamine, hexadecylamine, sphingosine and phytosphingosine. Examples of useful aliphatic alcohols include octanol, nonanol, decanol, dodecanol, hexadecanol, chimyl alcohol and selachyl alcohol.

Useful terpenoids include retinol, retinal, bisabolol, citral, citronellal, citronellol and linalool.

Useful mono- and di-acylglycerols include mono-, and di-esterified glycerols, wherein the acyl groups include from 8 to 20 carbon atoms, including 8 to 16 carbon atoms.

Useful polyethylene glycols have the formula H—(O—CH₂—CH₂)ₙ—OH, where n, the number of ethylene oxide units, is from 4 to 14. Examples of useful polyethylene glycols include PEG 200 (n=4), PEG 400 (n=8-9), and PEG 600 (n=12-14).

Useful polyethylene glycol fatty alcohol ethers, wherein the ethylene oxide units (n) are between 1 to 8, and the alkyl group is from C₆ to C₁₈.

A side-chain with amphipathic characteristics, i.e. asymmetric distribution of hydrophilic and hydrophobic groups, may facilitate e.g. the accessibility of a triterpene aldehyde to a cellular receptor. It is also possible that the presence of a negatively-charged carboxyl group in such a side-chain may contribute to the repulsion of the triterpene groups, thus allowing them a greater degree of rotational freedom. This last factor would most likely increase the accessibility of cellular receptors to the imine-forming carbonyl group.

In one preferred embodiment, when a saponin is linked to a lipophilic moiety, or to a bioactive agent, including a genetic determinant, by preparation of an active ester of glucuronic acid, a saponin component, followed by reaction of the active ester with a nucleophilic functional group on the lipophilic moiety, or the bioactive agent including a genetic determinant, such active esters may include the glucuronate of N-hydroxysuccinimide, sulfo-N-hydroxysuccinimide, hydroxybenzotriazole, and p-nitrophenol. The active esters may be prepared by reaction of the carboxy group of the saponin with an alcohol in the presence of a dehydration agent such as dicyclohexylcarbodiimide (DCC), 1-(3-dimethylaminopropyl)-3-ethylcarbodiimide (EDC), and 1-(3-dimethylaminopropyl)-3-ethylcarbodiimide methiodide (EDCI).

The use of EDC to form conjugates is disclosed in U.S. Pat. No. 4,526,714 to Feijen et al. and PCT application publication no. WO91/01750, and Arnon, R et al., Pros. Natl. Acad. Sci. (USA) 77:6769-6772 (1980), the disclosures of which are fully incorporated by reference herein. The bioactive agent including a genetic determinant is then mixed with the activated ester in aqueous solution to give the conjugate.

Where a linker group between the saponin and the bioactive agent including a genetic determinant is desired, the active ester of the saponin glucuronate is prepared as described above and reacted with the linker group, e.g. 2-aminoethanol, an alkylene diamine, an amino acid such as glycine, or a carboxy-protected amino acid such as glycine tert-butyl ester.

If the linker contains a protected carboxy group, the protecting group is removed and the active ester of the linker is prepared (as described above). The active ester is then reacted with the bioactive agent including a genetic determinant to give the conjugate. Alternatively, the bioactive agent including a genetic determinant may be derivatized with succinic anhydride to give an antigensuccinate conjugate which may be condensed in the presence of EDC or EDCI with a saponin-linker derivative having a free amino or hydroxyl group on the linker, as described in WO91/01750.

It is also possible to prepare a saponin conjugate comprising a linker with a free amino group (derived from an alkylene diamine) and crosslink the free amino group with a heterobifunctional cross linker such as sulfosuccinimidyl 4-(N-maleimido-cyclohexane)-1-carboxylate which will react with e.g. a free sulfhydryl group of a bioactive agent including a genetic determinant, including any derivative thereof.

The saponin may also be coupled to a linker group by reaction of the aldehyde group of the quillaic acid residue with an amino linker to form an intermediate imine conjugate, followed by reduction with sodium borohydride or sodium cyanoborohydride. Examples of such linkers include amino alcohols such as 2-aminoethanol and diamino compounds such as ethylenediamine, 1,2-propylenediamine, 1,5-pentanediamine, 1,6-hexanediamine, and the like. The bioactive agent including a genetic determinant may then be coupled to the linker by first forming the succinated derivative with succinic anhydride followed by condensation with the saponin-linker conjugate with DCC, EDC or EDCI.

In addition, the saponin may be oxidized with periodate and the dialdehyde produced therefrom condensed with an amino alcohol or diamino compound listed above. The free hydroxyl or amino group on the linker may then be condensed with the succinate derivative of the bioactive agent including a genetic determinant in the presence of DCC, EDC or EDCI.

Further useful linker groups are known in the art and examples are disclosed in e.g. U.S. Pat. No. 6,080,725, which is incorporated herein by reference.

One preferred contacting group is a cationic compound or group comprising at least one positively charged moiety at pH=7.0. Such moieties may e.g. be found in both saponins and sterols, and they may be found in lipophilic moities according to the present invention.

Examples include, but is not limited to quarternary ammonium compounds; dialkyl-dimethylammonium compounds; dioctadecyldimethyl ammonium chloride; dioctadecyldimethyl ammonium bromide; dioctadecyl/octadienyldimethyl ammonium chloride; dioctadecyl/octadienyldimethyl ammonium bromide; dimethyl-dioctadecyl ammonium bromide (DDAB), dodecyltrimethylammonium bromide, hexadecyltrimethylammonium compounds, mixed alkyltrimethylammonium bromide (Cetrimide per BP); and tetradecyltrimethylammonium compounds.

Additionally preferred contacting groups includes, but is not limited to, compounds comprising an essentially planar group that is capable of forming an intercalation between stacked bases of nucleic acids, including single stranded DNA (ssDNA), double stranded DNA (dsDNA), single stranded RNA (ssRNA), double stranded RNA (dsRNA), RNA and/or DNA comprising both a single stranded part and a double stranded part, small nuclear RNA (snRNA), hetroduplexes of RNA and DNA, including hetroduplexes of RNA and DNA comprising both a single stranded part as well as a double stranded part, peptide nucleic acids (PNA), locked nucleic acids (LNA), and the like. Examples of such contacting groups include, but is not limited to, acridines and phenanthridines, and derivatives thereof, cumarins, furocumarins, phytoalexins (e.g. psoralens), and derivatives thereof. Such contacting groups may occur either individually in a complex or in any combination with one or more additional contacting groups capable of forming an intercalation between stacked bases of nucleic acids as described herein above.

Further examples of preferred contacting groups includes, but is not limited to, indoles and imidazoles, including compounds such e.g. 4′,6-diamidio-2-phenylindole, 4′,6-(diimidazolin-2-yl)-2-phenylindole) (obtainable as Hoechst 33258, and Hoechst 33342, respectively), Actinomycin D (such as e.g. 7-Aminoactinomycin D); Cyanine dyes, dimers of cyanine dyes (such as e.g. TOTO (R), YOYO (R), BOBO (TM), POPO (TM)) and derivatives thereof.

Any compound having an affinity for a nucleic acid moiety can be used as a contacting group in accordance with the present invention. Accordingly, in addition to chelating groups, non-intercalating contacting groups can also be used. An example is hydroxystilbamidine (Fluoro-Gold (TM)) and derivatives hereof.

A comprehensive list of contacting groups capable of contacting nucleic acids and/or having an affinity to nucleic acids can be found e.g. in “Handbook of Fluorescent Probes and Research Chemicals”, by Richard P. Haugland, Sixth Edition, Molecular Probes (c) 1996, chapter 8, “Nucleic Acid Detection”. The compounds listed in the Haugland reference can be readily modified by the skilled person exerting nothing more than ordinary skill in the art in order to obtain derivatives and analogoues of said compounds listed therein.

Additionally preferred contacting groups are peptides and polypeptides including proteins, enzymes, co-enzymes, antibodies or binding fragments of antibodies having an affinity to nucleic acids, including the nucleic acids listed herein immediately above. Examples include, but is not limited to, nucleic acid binding proteins, including DNA binding proteins and proteins comprising a helix-turn-helix motif, including an alpha-helix—beta-turn—alpha-helix motif associated with the binding of DNA, Bacteriphage T4 gene 32 protein, E. coli single-stranded binding protein, RecA and homologues thereof, including E. coli RecA protein, Cytochrome C, monoclonal antibodies, Fab′ fragments of antibodies, and polyclonal antibodies.

Contacting groups may also be any nucleic acid capable of forming an association with another nucleic acid, and an analogous compound, including PNA and LNA, or a derivative thereof. The association may be formed by hydrogen bonding or any other interaction resulting in base-pairing and/or duplex formation and/or triplex formation with at least one genetic determinant. Examples include oligonucleotides and oligonucleotides modified with lipophilic compounds.

As described herein above, lipophilic moieties may serve the purpose of facilitating complex formation while at the same time acting as a “docking” group for contacting groups and/or targeting ligands. The lipophilic moieties may thus form part of the complexes according to the present invention.

Lipophilic moieties according to the present invention are any moiety, including any residue of a lipophilic molecule, which is attached to or in contact with either i) any suitable functional group of one or more compounds that is essentially non-polar, or ii) forms an essentially non-polar domain within the complexes according to the present invention.

The lipophilic moiety can be a portion of an amphipathic compound. An amphipathic compound is a compound whose molecules contain both polar and non-polar domains. Surfactants are examples of amphipathic compounds. Surfactants typically possess a non-polar portion that is often an alkyl, aryl or terpene structure. In addition, a surfactant possesses a polar portion, that can be anionic, cationic, amphoteric or non-ionic. Examples of anionic groups are carboxylate, phosphate; sulfonate and sulfate. Examples of cationic domains are amine salts and quaternary ammonium salts. Amphoteric surfactants possess both an anionic and a cationic domain. Non-ionic domains are typically derivatives of a fatty acid carboxy group and include saccharide and polyoxyethylene derivatives.

A lipophilic moiety can also comprise two or more compounds possessing non-polar domains, wherein each of the compounds has been bonded to a linking group, which, in turn, is covalently attached to a component of the complex according to the present invention, including any saponin component and/or any sterol component comprised in said complex, including any residues of said sterol component and any residues of said saponin component, including any aglycone part and/or any saccharide part.

One group of preferred lipophilic moieties are phospholipids such as phosphatidylcholine, phosphatidylethanolamine, triglycerides, fatty acids, and hydrophobic amino acids residues including membrane spanning hydrophobic amino acid segments.

When the complexes according to the present invention comprises i) a saponin derived from a Quil A fraction as described by WO 92/06710, which is incorporated herein by reference, ii) cholesterol and iii) phosphatidylcholine or phosphatidylethanolamine, the complex further comprises at least one bioactive agent, including a genetic determinant, including a polynucleotide, including any derivative thereof as described herein. In one embodiment, such complexes have a ratio (weight per weight) of i) lipid and cholesterol to ii) saponin, of more than 1:2, such as more than 1.5:2, and a lipid concentration of more than 1 mg/ml, for example more than 1.2 mg/ml.

Examples of lipophilic moieties capable of being used in connection with the present invention are lipids other than sterols, for example fats or fat resembling substances such as e.g. triglycerides or mixed triglycerides containing fatty acids with up to 50 carbon acids such as saturated fatty acids having for example 4, 5, 6, 7, 8, 9, 10, 11, 12, 13, 14, 15, 16, 17, 18, 19, 20, 21, 22, 23, 24, 25, 26, 27, 28, 29 and 30 carbon atoms e.g. burytic acid, caprole acid, caprylic acid, captic acid, lauric acid, myristic acid, palmitic acid, stearic acid, arachidic acid, behenic acid, lignoceric acid; unsaturated fatty acids with up to 30 carbon atoms, such as hexadecene acid, oleic acid, linoleic acid, linolenic acid, arachidonic acid; hydroxy-fatty acids such as 9,10-dihydroxy stearic acid; unsaturated hydroxy fatty acids such as castor oil; branched fatty acids such as glycerol ethers; waxes i.e. esters between higher fatty acids and monohydric alcohols; phospholipides such as derivatives of glycerol phosphates such as derivatives of phosphatidic acids i.e. lecithin, cephalin, inositol phosphatides, spingosine derivatives with 14, 15, 16, 17, 18, 19 and 20 carbon atoms; glycolipids; isoprenoids; sulpholipids; and carotenoids.

Additional examples of lipophilic moieties capable of forming part of the complexes according to the present invention are cationic lipids. It will be understood that cationic lipids according to the definition applied herein are lipids carrying a net positive charge at pH 7.0.

Cationic lipids which may be used in the compositions of the present invention include, for example, phosphatidyl ethanolamine, phospatidyl choline, glycero-3-ethylphosphatidyl choline and fatty acyl esters thereof, di- and trimethyl ammonium propane, di- and tri-ethylammonium propane and fatty acyl esters thereof. A preferred derivative from this group is N-[1-(2,3-dioleoyloxy)propyl]-N,N,N-trimethylammonium chloride (“DOTMA”).

Additionally, a wide array of synthetic cationic lipids can function in the present invention. These include common natural lipids derivatized to contain one or more basic functional groups. Examples of lipids which can be so modified include, for example, dimethyldioctadecylammonium bromide, sphingolipids, sphingomyelin, lysolipids, glycolipids such as ganglioside GM1, sulfatides, glycosphingolipids, cholesterol and cholesterol esters and salts, N-succinyldioleoyl-phosphatidyl ethanolamine, 1,2,-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidyl ethanolamine and palmitoylhomocystiene.

In one embodiment, the cationic lipid in the composition of the present invention is a fluorinated cationic lipid. Any of the cationic lipids described herein may be fluorinated by replacing at least one hydrogen atom with a fluorine atom

Specially synthesized cationic lipids also function in the present invention, including those compounds of formula (I), formula (II) and formula (III), described in U.S. Pat. No. 6,120,751, the disclosure of which is hereby incorporated by reference herein in its entirety.

Further examples of lipophilic moieties capable of forming part of complexes according to the present invention are, for example, N,N′-Bis (dodecyaminocarbonyl-methylene)-N,N′-bis (β-N,N,N-trimethylammoniumethyl-aminocarbonyl-methylene)-ethylenediamine tetraiodide; N,N″-Bis (hexadecylamino-carbonyl-methylene)-N,N′,N″-tris(β-N,N,N-trimethylammoniumethylaminocarbonyl-methylenediethylene-triamine hexaiodide; N,N′-Bis (dodecylaminocarbonylmethylene)-N,N″-bis(β-N,N,N-trimethylammoniumethylaminocarbonylmethylene)cyclohexylene-1,4-diamine tetraiodide; 1,1,7,7-tetra-(β-N,N,N,N-tetramethylammoniumethylamino-carbonylmethylene)-3-hexadecylaminocarbonylmethylene-1,3,7-triaazaheptane heptaiodide; and N,N,N′N′-tetra (β-N,N,N-trimethylammoniumethylaminocarbonylmethylene)-N′-(1,2-dioleoylglycero-3-phosphoethanolaminocarbonylmethylene)-diethylenetriamine tetraiodide.

In one preferred embodiment, the cationic lipid is a fluorinated cationic lipid. Any of the cationic lipids described herein may be fluorinated by replacing at least one hydrogen atom with a fluorine atom. One skilled in the art will recognize that countless other natural and synthetic variants carrying positive charged moieties will also function in the invention.

In addition to the cationic lipids described above, other suitable lipids which may be used in the present invention include, for example, fatty acids, lysolipids, fluorinated lipids, phosphocholines, such as those associated with platelet activation factors (PAF) (Avanti Polar Lipids, Alabaster, Ala.), including 1-alkyl-2-acetoyl-sn-glycero 3-phosphocholines, and 1-alkyl-2-hydroxy-sn-glycero 3-phosphocholines, which target blood clots; phosphatidylcholine with both saturated and unsaturated lipids, including dioleoylphosphatidylcholine; dimyristoylphosphatidylcholine (DMPC); dipentadecanoylphosphatidylcholine; dilauroylphosphatidylcholine; dipalmitoylphosphatidylcholine (DPPC); distearoylphosphatidylcholine (DSPC); and diarachidonylphosphatidylcholine (DAPC); phosphatidylethanolamines, such as dioleoylphosphatidylethanolamine, dimyristoylphosphatidylethanolamine (DMPE), dipalmitoylphosphatidylethanolamine (DPPE) and distearoylphosphatidylethanolamine (DSPE); phosphabidylserine; phosphatidylglycerols, including distearoylphosphatidylglycerol (DSPG); phosphatidylinositol; sphingolipids such as sphingomyelin; glycolipids such as ganglioside GM1 and GM2; glucolipids; sulfatides; glycosphingolipids; phosphaidic acids, such as dipalmitoylphosphatidic acid (DPPA) and distearoylphosphatidic acid (DSPA); palmitic acid; stearic acid; arachidonic acid; oleic acid; linolenic acid; linoleic acid; myristic acid; synthetic lipids described in U.S. Pat. No. 5,312,617, the disclosure of which is hereby incorporated by reference herein in its entirety.

Additionally preferred lipophilic moieties include, but is not limited to glycolipids, phosphatidylethnolamine, phosphatidylcholine, phosphatydilinositol, phosphatidylserine, phosphatidylglycerol, including derivatives thereof. Further preferred lipophilic moieties are sphingomyelin, diphosphatidylglycerol (Cardiolipin), phosphatidic acid, Tfx™ Reagents, including Tfx™-10 Reagent, Tfx™-20 Reagent, and Tfxυ-50 Reagent, and 1,2-Diacyl-sn-Glycero-3-Ethylphosphocholine compounds, particular compounds wherein the acyl groups, independently from another, is selected from the group consisting of lauroyl, myristoyl, palmitoyl, stearoyl, oleoyl, palmitoyl-oleoyl, and of dilauroyl, dimyristoyl, dipalmitoyl, distearoyl, dibleoyl (DOPC+). L-α-Dioleoyl-Phosphatidylethanolamine, or 1,2-dioleoyl-sn-glycero-3-phosphoethanolamine (DOPE) represents one particularly preferred lipophilic moiety.

Additionally preferred are DOTAP; DDAB (dimethyl dioctadecylammonium bromide); 1,2-Dioleoyl-sn-glycero-3-phosphoethanolamine DOPE; L-β,γ-Dioleoyl-α-cephalin; 3-sn-Phosphatidylethanolamine, 1,2-dideoyl N-(1-[2,3-Dioleoyloxy]propyl)-N,N,N-trimethylammonium; Dioctadecyl dimethyl ammonium bromide; Avridine (CP-20,961), and stearyl tyrosine.

One particularly interesting lipophilic moiety is Monophosphoryl lipid A (MPL) as described by Baldridge and Crane (1999) in Methods, vol. 19, no. 1, p. 103-107; by Zhou and Huang (1993) in Vaccine, vol. 11, no. 11, p. 1139-1144 and by Rudbach et al. (1995) in Chap. 13. in “The Theory and Practical Application of Adjuvants” (Stewart-Tull, ed), Wiley & Sons, Ltd. Cationic derivatives of MPL are also included in the present invention. MPL can be obtained from Corixa Corp. (www.corixa.com).

Additionally preferred lipolytic moieties are lipids bearing polymers, such as chitin, hyaluronic acid, polyvinylpyrrolidone or polyethylene glycol (PEG), also referred to herein as “pegylated lipids” with preferred lipid bearing polymers including DPPE-PEG (DPPE-PEG), which refers to the lipid DPPE having a PEG polymer attached thereto, including, for example, DPPE-PEG5000, which refers to DPPE having attached thereto a PEG polymer having a mean average molecular weight of about 5000; lipids bearing sulfonated mono-, di-, oligo- or polysaccharides; cholesterol, cholesterol sulfate and cholesterol hemisuccinate; tocopherol hemisuccinate; lipids with ether and ester-linked fatty acids; polymerized lipids (a wide variety of which are known in the art); diacetyl phosphate; dicetyl phosphate; stearylamine; cardiolipin; phospholipids with short chain fatty acids of about 6 to about 8 carbons in length; synthetic phospholipids with asymmetric acyl chains, such as, for example, one acyl chain of about 6 carbons and another acyl chain of about 12 carbons; ceramides; non-ionic liposomes including niosomes such as polyoxyalkylene (e.g., polyoxyethylene) fatty acid esters, polyoxyalkylene (e.g., polyoxyethylene) fatty alcohols, polyoxyalkylene (e.g., polyoxyethylene) fatty alcohol ethers, polyoxyalkylene (e.g., polyoxyethylene) sorbitan fatty acid esters (such as the class of compounds referred to as TWEEN.RTM., including, for example, TWEEN.RTM. 20, TWEEN.RTM. 40 and TWEEN.RTM. 80, commercially available from ICI Americas, Inc., Wilmington, Del.), glycerol polyethylene glycol oxystearate, glycerol polyethylene glycol ricinoleate, alkyloxylated (e.g., ethoxylated) soybean sterols, alkyloxylated (e.g., ethoxylated) castor oil, polyoxyethylene-polyoxy-propylene polymers, and polyoxyalkylene (e.g., polyoxyethylene) fatty acid stearates; sterol aliphatic acid esters including cholesterol sulfate, cholesterol butyrate, cholesterol isobutyrate, cholesterol palmitate, cholesterol stearate, lanosterol acetate, ergosterol palmitate, and phytosterol-n-butyrate; sterol esters of sugar acids including cholesterol glucuronide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including, glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, and n-octadecyl alcohol; 6-(5-cholesten-3β-yloxy)-1-thio-β-D-galacto-pyranoside; digalactosyldiglyceride; 6-(5-cholesten-3β-yloxy)-hexyl-6-amino-6-deoxy-1-thio-β-D-galactopyranoside; 6-(5-cholesten-3β-yloxy)hexyl-6-amino-6-deoxyl-1-thio-α-D-mannopyranoside; 12-(((7′-diethylaminocoumarin-3-yl)-carbonyl)methylamino)-octadecanoic acid; N-[12-(((7′-diethylaminocoumarin-3-yl)-carbonyl)methylamino)-octadecanoyl]-2-aminopalmitic acid; cholesteryl(4′-trimethylammonio)butanoate; N-succinyldioleoylphosphatidyl-ethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycero-phospho-ethanolamine and palmitoylhomocysteine, and/or any combinations thereof.

One skilled in the art could readily determine the charge (e.g., cationic, anionic or neutral) of any of the lipids described herein. In a preferred embodiment, the lipids described herein are fluorinated lipids. As one skilled in the art will recognize, any of the neutral lipids described herein may be modified to cationic lipids or anionic lipids by methods that are well-known to one skilled in the art. For example, any modifiable group on a neutral lipid, such as a secondary amine, an —OH group or an anionic group or cationic group that have a zwitterionic charge balance, may be chemically modified to add or subtract a charge to the neutral lipid.

When a neutral lipid is used in the compositions of the present invention, the neutral lipid is preferably a phosphocholine, a sphingolipid, a glycolipid, a glycosphingolipid, a phospholipid or a polymerized lipid.

Examples of polymerized lipids include unsaturated lipophilic chains such as alkenyl or alkynyl, containing up to about 50 carbon atoms. Further examples are phospholipids such as phosphoglycerides and sphingolipids carrying polymerizable groups; and saturated and unsaturated fatty acid derivatives with hydroxyl groups, such as for example triglycerides of d-1,2-hydroxyoleic acid, including castor oil and ergot oil.

Polymerization may be designed to include hydrophilic substituents such as carboxyl or hydroxyl groups, to enhance dispersability so that the backbone residue resulting from biodegradation is water soluble. Suitable polymerizable lipids are also described, for example, by Klaveness et al, U.S. Pat. No. 5,536,490, the disclosure of which is hereby incorporated by reference herein in its entirety.

Even further examples of lipophilic moieties capable of forming part of complexes according to the present inventon are those e.g. described in EP 0 109 952 B1, incorporated herein by reference, and in EP 0 436 620 B1, incorporated herein by reference. In particular, the lipophilic moiety may be a phospholipid such as phosphatidyl-ethanolamine and phosphatidylcholine.

The lipophilic moiety may comprise a lipophilic receptor molecule capable of binding a cell-binding component such as e.g. an antigen. Examples of such receptors are e.g. glycolipids, for example the cholera toxin's receptor ganglioside GM1 and fucosylated blood group antigen. The cell-binding component can then function as a transport molecule.

In one embodiment, the lipophilic moiety of the complex according to the invention is covalently bonded to at least one polymer including, for example, hydrophilic polymers. Suitable hydrophilic polymers for covalent bonding to lipids include, for example, polyalkyleneoxides such as, for example, polyethylene glycol (PEG) and polypropylene glycol (PPG), polyvinyl-pyrrolidones, polyvinylalkylethers, such as a polyvinylmethyl ether, polyacrylamides, such as, for example, polymethacrylamides, polydimethyl-acrylamides and polyhydroxy-propylmethacrylamides, polyhydroxyakyl(meth)-acrylates, such as polyhydroxyethyl acrylates, polyhydroxypropyl methacrylates, polyalkyloxazolines, such as polymethyloxazolines and polyethyloxazolines, polyhydroxyalkyloxazolines, such as polyhydroxyethyloxazolines, polyhyhydroxypropyloxazolines, polyvinyl alcohols, polyphosphazenes, poly(hydroxyalkylcarboxylic acids), polyoxazolidines, polyaspartamide, and polymers of sialic acid (polysialics).

Preferably, the hydrophilic polymers are polyethylene glycol, polyvinyl pyrrolidone, polyvinyl alcohol, polypropylene glycol, a polyvinylalkylether, a polyacrylamide, a polyalkyloxazoline, a polyhydroxyalkyloxazoline, a polyphosphazene, a polyoxazolidine, a polyaspartamide, a polymer of sialic acid, a polyhydroxyalkyl(meth)acrylate or a poly(hydroxyalkylcarboyxlic acid).

More preferably, the hydrophilic polymers are PEG, PPG, polyvinylalcohol, polyvinylpyrrolidone and copolymers thereof, with PEG and PPG polymers being more preferred and PEG polymers being even more preferred. The polyethylene glycol may be, for example, PEG 2000, PEG 5000 or PEG 8000, which have weight average molecular weights of 2000, 5000 and 8000 daltons, respectively.

Preferably, the polyethylene glycol has a molecular weight of about 500 to about 20,000, more preferably from about 1,000 to about 10,000. Other suitable polymers, hydrophilic and otherwise, will be apparent to one skilled in the art based on the present disclosure.

Exemplary lipids which are covalently bonded to hydrophilic polymers include, for example, dipalmitoylphosphatidylethanolamine-PEG, dioleoylphosphatidylethanolamine-PEG and distearylphosphatidylethanolamine-PEG, more preferably dipalmitoylphosphatidylethanolamine-PEG.

Posintros with a specific size frequently target preferred tissues. The posintros according to the present invention may be selected according to size, wherein the specific size of the posintro may aid targeting of the targeting vehicle to specific tissues.

In addition to the herein above described targeting vehicles, the targeting vehicle according to the present invention may also be any other suitable targeting vehicle. In one embodiment the targeting vehicle comprises an ISCOM.

In another embodiment the targeting vehicle comprises a liposome. A liposome within the meaning of the present invention is generally spherical or spheroidal cluster or aggregate of amphipathic compounds, including lipophilic moieties, typically in the form of one or more concentric layers, for example, monolayers, bilayers or multi-layers. They may also be referred to herein as lipid vesicles. The liposomes may be formulated, for example, from ionic lipids and/or non-ionic lipids. Liposomes formulated from non-ionic lipids may be referred to as niosomes. Liposomes formulated, at least in part, from cationic lipids or anionic lipids may be referred to as cochleates.

The liposomes may be prepared e.g. as described by Lipford and Wagner (1994) in Vaccine, vol. 12, no. 1, p. 73-80, incorporated herein by reference. General liposomal preparatory techniques which may be adapted for use in the preparation of liposome compositions pertaining to the present invention are discussed, for example, in U.S. Pat. Nos. 4,728,578, 4,728,575, 4,737,323, 4,533,254, 4,162,282, 4,310,505, and 4,921,706; U.K. Patent Application GB 2193095A; International Application Serial Nos. PCT/U.S.85/01161 and PCT/U.S.89/05040; Mayer et al., Biochimica et Biophysica Acta, 858:161-168 (1986); Hope et al., Biochimica et Biophysica Acta, 812:5565 (1985); Mayhew et al., Methods in Enzymology, 149:64-77 (1987); Mayhew et al., Biochimica et Biophysica Acta, 755:169-74 (1984); Cheng et al, Investigative Radiology, 22:47-55 (1987); and Liposome Technology, Gregoriadis, G., ed., Vol. I, pp. 29-31, 51-67 and 79-108 (CRC Press Inc., Boca Raton, Fla. 1984), the disclosures of each of which are hereby incorporated by reference herein.

Accordingly, the liposome compositions of the invention may be prepared using any one of a variety of conventional liposomal preparatory techniques which will be apparent to one skilled in the art, including, for example, solvent dialysis, French press, extrusion (with or without freeze-thaw), reverse phase evaporation, simple freeze-thaw, sonication, chelate dialysis, homogenization, solvent infusion, microemulsification, spontaneous formation, solvent vaporization, solvent dialysis, French pressure cell technique, controlled detergent dialysis, and others, each involving the preparation of the compositions in various fashions. See, e.g., Madden et al., Chemistry and Physics of Lipids, 53:3746 (1990), the disclosure of which is hereby incorporated herein by reference.

Suitable freeze-thaw techniques are described, for example, in International Application Serial No. PCT/U.S.89/05040, filed Nov. 8, 1989, the disclosure of which is hereby incorporated herein by reference in its entirety. Methods. which involve freeze-thaw techniques are preferred in connection with the preparation of liposomes. Preparation of the liposomes may be carried out in a solution, such as an aqueous saline solution, aqueous phosphate buffer solution, or sterile water. The liposomes may also be prepared by various processes which involve shaking or vortexing, which may be achieved, for example, by the use of a mechanical shaking device, such as a Wig-L-Bug.TM. (Crescent Dental, Lyons; Ill.), a Mixomat (Degussa AG Frankfurt, Germany), a Capmix (Espe Fabrik Pharmazeutischer Praeparate GMBH & Co., Seefeld, Oberay Germany), a Silamat Plus (Vivadent, Lechtenstein), or a Vibros (Quayle Dental, Sussex, England). Conventional microemulsification equipment, such as a Microfluidizer.TM. (Microfluidics, Woburn, Mass.) may also be used.

In one embodiment of the invention the targeting vehicle comprises a biodegradable microsphere. In another embodiment the targeting vehicle comprises an encapsulation system. In one preferred embodiment the targeting vehicle comprises a cochleate. In yet another embodiment the targeting vehicle comprises a nanoparticle. In a still further embodiment the targeting vehicle comprises a hydrogel. In an even still further embodiment the targeting vehicle comprises a microcrystal.

Targeting

Targeting to specific cells may be achieved in a number of different ways. The simplest method is by administration directly to the site of the target cells. For example, if the foreign immunogen and/or foreign antigen is a peptide, large amounts of said peptide may be administrated directly to the site of the target cells. This may result in peptide replacement, such as peptides which are presented by the MHC molecules of the target cells, may be replaced by the administrated peptides.

Alternatively, the foreign immunogen and/or antigen may be taken up by target cells after administration to the site of the target cells by unspecific mechanisms.

In one embodiment the foreign immunogen and/or antigen may be associated with a targeting vehicle, which may facilitate the uptake by the targeted cells by mechanisms unspecific to the immunogen/antigen. A preferred example hereof is lipopeptides embedded into ISCOMs or embedded into posintro. Examples of posintros are given herein above.

In one preferred embodiment the foreign immunogen and/or the foreign antigen is a nucleic acid encoding a polypeptide or peptide and said nucleic acid is associated with posintro. Alternatively other delivery systems for nucleic acids may be employed.

Selecting a targeting vehicle with suitable physical properties, may aid targeting to the correct tissue. For example, the size of the targeting vehicle may determine preferred target tissues.

In one embodiment of the present invention the vehicle comprises a lipid, which can associate with lipid rafts of the target cells. Association with lipid rafts may furthermore aid internalisation of the targeting complex.

Examples of lipophilic compounds are given herein above.

In a preferred embodiment the vehicle comprises a specific binding partner. A specific binding partner is a molecule, which specifically may interact with another molecule, which is associated with the cell surface via a biospecifc interaction. Preferably, the molecule associated with the cell surface is primarily associated with the cell surface of the target cells and absent or present at reduced levels in other cells.

Preferably, the molecule associated with the cell surface is expressed on the surface of the target cells at a level more than 1.5 fold, such as more than 2 fold, for example more than 3 fold, such as more than 4 fold, for example more than 5 fold, such as more than 6 fold, for example more than 7 fold, such as more than 8 fold, for example more than 9 fold, such as more than 10 fold, for example more than 15 fold, such as more than 20 fold, for example more than 30 fold, such as more than 50 fold, for example more than 100 fold, such as more than 250 fold, for example more than 500 fold, such as more than 750 fold, for example more than 1000 fold higher than level of expression on other cells.

In one embodiment the specific binding partner may be capable of being internalised. Preferably, the specific binding partner may be taken up by target cells by receptor mediated endocytosis. However, specific binding partners, which are not capable of being internalised should also be regarded as part of the present invention.

The specific binding partner may for example comprise a polypeptide, a peptide or a small chemical compound. The specific binding partner may also essentially consist of or consist of a polypeptide, a peptide or a small chemical compound.

In one embodiment the specific binding partner comprise an antibody or a binding fragment of an antibody. The antibody may be polyclonal antibody or a monoclonal antibody. The antibody may be derived from any species, preferably a mammal. Alternatively, the antibody may be a synthetic antibody or a chimeric antibody, which comprises fragments derived from different species.

The specific binding partner according to the present invention, preferably is capable forming a biospecific interaction with a molecule, which is associated the cell surface.

For example specific binding partners may be ligands of the family of G protein-coupled receptors or derivatives thereof or compounds mimicking said ligands.

Preferred G protein-coupled receptors according to the present invention may be selected from the group consisting of members of the following subfamilies (nomenclature is according to “GPCRDB: Information system for G protein-coupled receptors (GPCRs)” available on the Internet at http://www.qpcr.orq/7tm/ (Horn et al, Nucleic Acids Research, 2001, 29, 1 346-349):

Acetylcholine

Adenosine type 1

Adenosine type 2

Adenosine type 3

Adrenocorticotropic hormone

Adrenomedullin (G10D)

Alpha Adrenoceptors type 1

Alpha Adrenoceptors type 2

Angiotensin type 1

Angiotensin type 2

APJ like

Beta Adrenoceptors type 1

Beta Adrenoceptors type 2

Beta Adrenoceptors type 3

Beta Adrenoceptors type 4

Bombesin

BONZO

Bradykinin

Brain-specific angiogenesis inhibitor (BAI)

C5a anaphylatoxin

Calcitonin

Cannabis

C—C Chemokine other

C—C Chemokine type 1

C—C Chemokine type 10

C—C Chemokine type 2

C—C Chemokine type 3

C—C Chemokine type 4

C—C Chemokine type 5

C—C Chemokine type 6

C—C Chemokine type 7

C—C Chemokine type 8

C—C Chemokine type 9

C—C Chemokine type 9/10

CCK type A

CCK type B

Chemokine receptor-like 2

Chemokine/chemotactic factors like

Class B orphan/other

Class E cAMP receptors (Dictyostelium)

Conopressin

Corticotropin releasing factor

C—X3-C Chemokine

C—X—C Chemokine type 3

C—X—C Chemokine type 4

C—X—C Chemokine type 5

Diuretic hormone

Dopamine Other

Dopamine Vertebrate type 1

Dopamine Vertebrate type 2

Dopamine Vertebrate type 3

Dopamine Vertebrate type 4

EBV-induced

EMR1

Endothelin

Extracellular calcium-sensing

Fmet-leu-phe

Follicle stimulating hormone

frizzled Group A (Fz 1&2&4&5&7-9)

frizzled Group B (Fz 3 & 6)

frizzled Group C (other)

GABA-B subtype 1

GABA-B subtype 2

Galanin

Gastric inhibitory peptide

Glucagon

Gonadotropin

Gonadotropin-releasing hormone

GP40 like

GPR

GPR37 like (peptide receptor)

Growth hormone secretagogue

Growth hormone secretagogue like

Growth hormone-releasing hormone

Histamine type 1

Histamine type 2

Interleukin-8 other

Interleukin-8 type A

Interleukin-8 type B

Latrotoxin type 1

Latrotoxin type 2

Latrotoxin type 3

Lutropin-choriogonadotropic hormone

Lysosphingolipid & LPA (EDG)

Mas proto-oncogene

Melanocortin hormone

Melanocyte stimulating hormone

Metatonin

Metabotropic glutamate group I

Metabotropic glutamate group III

Metabotropic glutamate other

Neuromedin K (NK3)

Neuropeptide Y/peptide YY

Neuropeptide Y other

Neuropeptide Y type 1

Neuropeptide Y type 2

Neuropeptide Y type 4

Neuropeptide Y type 5

Neuropeptide Y type 6

Neurotensin

Octopamine

Ocular albinism proteins

Olfactory FOR-like (fish)

Olfactory MOR-like (mouse)

Olfactory other

Olfactory type 1

Olfactory type 10

Olfactory type 2

Olfactory type 3

Olfactory type 5

Olfactory type 6

Olfactory XOR-like (frog)

Opioid type D

Opioid type K

Opioid type M

Opioid type X

Orexin

ORPH

Orphan GPRC5

Oxytocin

PACAP

Parathyroid hormone

Plant MIo receptors

Platelet activating factor

Prostacyclin

Prostaglandin E2 subtype EP1

Prostaglandin E2 subtype EP3

Prostaglandin E2 subtype EP4

Prostaglandin E2/D2 subtype EP2

Prostaglandin F2-alpha

Proteinase activated

Purinoceptor Other

Purinoceptor type U

Purinoceptor type Y adenine

Purinoceptor type Y adenine/uridine

Purinoceptor type Y uridine

Putative/unclassified Class A GPCRs

Putative/unclassified Class B GPCRs

Putative/unclassified Class C GPCRs

Putative/unclassified GPCRs

Putative pheromone Receptors

RDC1

Rhodopsin Mollusc

Rhodopsin Other

Rhodopsin Vertebrate type 1

Rhodopsin Vertebrate type 2

Rhodopsin Vertebrate type 3

Rhodopsin Vertebrate type 4

Rhodopsin Vertebrate type 5

Secretin

Serotonin Insect type 1

Serotonin Insect type 2

Serotonin Other

Serotonin Vertebrate type 1

Serotonin Vertebrate type 2

Serotonin Vertebrate type 4

Serotonin Vertebrate type 5

Serotonin Vertebrate type 6

Serotonin Vertebrate type 7

Smoothened

Somatostatin type 1

Somatostatin type 2

Somatostatin type 3

Somatostatin type 4

Somatostatin type 5

SREB

Substance K (NK2)

Substance P (NK1)

Tachykinin like 1

Tachykinin like 2

Tachykinin like 3

Thrombin

Thromboxane

Thyrotropin

Thyrotropin-releasing hormone

Urotensin II

Vasoactive intestinal polypeptide

Vasopressin

Viral

XC Chemokine

Ligands of the G protein-coupled receptors according to the present invention may be selected from the group consisting of:

8-OH-DPAT, Aminoketanserin, Atropine, Butaclamol, Chlorpromazine, Chlorprothixene, Cinanserin, Cyanopindolol, Cyproheptadine, Domperidone, Dopamine, Epi-depride, Epi-nephrine, Fenoldopam, Flupen thixol, Fluphenazine, Haloperidol, Hexocyclium, Himbacine, Iodomelatonin, Ketanserin, LSD, Mesoridazine, Mesulergine, Methoctramine, Methysergide, Metoclopramide, Mianserin, Molindonem, Muscarinic, Naloxone, N-Methylspiperone, Nor-epinephrine, Pergolide, Phentolamine, Pirenzepine, PPHT-coumarin, PPHT-rhodamine, PPHT-Texas red, Prazosin, Promazine, Raclopride, Serotonin, Spiperone, Spiroxatrine, Sulpiride, Sumatriptan, Tenilapine and Triflupromazine.

Furthermore, specific binding partners according to the present invention may be ligands of Ligand activated ion channels, derivatives of said ligands or compounds mimicking said ligands.

Ligands of Ligand activated ion channels for example be selected from the group consisting of acetylcholine, adenosine triphosphate, serotonin, GABA (g-amino butyric acid), glutamate and glycine.

Ligand activated ion channels may be selected from the group consisting of members of the superfamilies Nicotinicoid receptor superfamily, ATP gated channel superfamily and Glutamate cationic receptor superfamily.

The Nicotinicoid receptor superfamily for example comprise 5HT₃ receptors, nicotinic acetylcholine receptors, glycine receptors, GABA_{A} and GABA_{C} receptors and anionic glutamate receptors.

The Glutamate cationic receptor superfamily for example comprise AMPA receptors, kainate receptors and NMDA receptors.

In addition any of the Ligand activated ion channels and ligand thereof may be selected from the detailed listings of these ion channel complexes in “The Ligand Gated Ion Channel Database” compiled by Le Novèr et al. (Nucleic Acid Research, 1999, 27: 340-342).

In one preferred embodiment of the present invention the specific binding partner is a vitamin. Alternatively the specific binding partner may comprise a vitamin or fragments thereof or derivatives thereof.

Vitamins according to the present invention may be selected from the group consisting of vitamin A, vitamin B1, vitamin B2, vitamin B3, vitamin B5, vitamin B6, vitamin B10, vitamin B11, vitamin B12, vitamin B13, vitamin B15, vitamin B17, vitamin C, vitamin D, vitamin E, vitamin F, vitamin G, vitamin H, vitamin K, vitamin L, vitamin M, vitamin P, vitamin T and vitamin U. Preferably, the specific binding partner is a vitamin selected from the vitamin B family, more preferably, the specific binding partner is folic acid.

The targeting complex according to the present invention may further comprise a biologically active component. A biological active component may be any component, which directly or indirectly can influence the immune response of an individual. Preferably, the biological active component is selected from the group consisting of cytokines and chemokines and nucleic acid sequences encoding cytokines and chemokines.

Cytokines may for example be selected from the group consisting of IL-2, IL-4, IL-10, IL-12, IL-15, IL-18, IL-21; IFN-γ, IFN-α, IFN-β, GM-CSF, C-CSF.

Alternatively, the biological active component may be any component capable of inducing apoptosis in cells. In particular, the biologically active component may be a compound capable of inducing apoptosis in the targeted cell. Preferably, the biologically active component may be a member of the Pro-Apoptotic Bcl-2 Family of proteins or the biologically active component may be a subunit of the Apoptasome Complex or the biological active component may be a Caspase.

Other examples of biological active components may be any receptor or ligand capable of biasing immune cells acting specifically against the targeted cell.

Such receptors may be any receptor of the family of Cellular Differation antigens (CD), for example CDs may be selected from the group consisting of CD28, CD80 and CD86. Such ligands may be any ligand for the receptors of the family of Cellular Differentiation antigens (CD), for example ligands may be selected from the group consisting of CD40 ligand, L-selectin, CD27 ligand, CD30 ligand and CD70.

Of special interest for the present invention are biological active substances specific for dendricitc cells, viz. receptors and ligands for dendrictic cell surface molecules. Examples of such receptors and/or ligands are: CD11b and/or CD11b ligands, CD3 and/or CD3 ligands, CD13 and/or CD13 ligands, CD14 and/or CD14 ligands, DEC205 and/or ligands to DEC205, Flt3 (Fetal liver tyrosine-kinase 3 ligand) and/or receptors for Flt3, and/or EPSTI1 (epithelial-stromal interaction-1) and/or receptors or binding partners thereof.

The biologically active component may furthermore be any of the bioreactive species which are described in the international patent application PCT/DK02/00229.

In one embodiment of the present invention the targeting complex may be comprised within a vaccine formulation (see herein above).

Preferably, the present invention do not comprise in vitro steps, wherein in vitro steps may be selected from the group consisting of

i) cultivating cells of the individual in vitro

ii) manipulating cells of the individual in vitro

iii) manipulating other cells in vitro and administrating said cells to the individual

However, in certain embodiments of the present invention the steps of the present invention may be combined with one or more in vitro steps.

Conditions

The condition to be treated according to the present invention, is any condition characterised by the presence of cells capable of being targeted, which are desirable to target in order to treat the clinical conditions.

For example such a condition may be cancer. Cancer is characterised by the presence of undesirable malignant cells. Accordingly, it is an objective of the present invention to target the foreign antigen according to the invention to malignant cells of an individual with the targeting complex according to the present invention. Preferably, the targeted cells may be eliminated by the cytotoxic and/or inflammatory response enabled by the antigen.

The malignant cells to be targeted according to the present invention may be associated any cancer.

Cancer according to the present invention may be selected from the group consisting of colon carcinoma, breast cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangeosarcoma, lymphangeoendothelia sarcoma, synovioma, mesothelioma, Ewing's sarcoma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumour, cervical cancer, testicular tumour, lung carcinoma, small cell lung carcinoma, bladder carcinoma, epithelial carcinoma, glioblastomas, neuronomas, craniopharingiomas, schwannomas, glioma, astrocytoma, medulloblastoma, craniopharyngioma, ependymoma, pinealoma, hemangioblastoma, acoustic neuroama, oligodendroglioma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, Hodgkin's Disease, non-Hodgkin's lymphomas, rectum cancer, urinary cancers, uterine cancers, oral cancers, skin cancers, stomach cancer, brain tumours, liver cancer, laryngeal cancer, esophageal cancer, mammary tumours, childhood-null acute lymphoid leukemia (ALL), thymic ALL, B-cell ALL, acute myeloid leukemia, myelomonocytoid leukemia, acute megakaryocytoid leukemia, Burkitts lymphoma, acute myeloid leukemia, chronic myeloid leukemia, and T cell leukemia, small and large non-small cell lung carcinoma, acute granulocytic leukemia, germ cell tumours, endometrial cancer, gastric cancer, cancer of the head and neck, chronic lymphoid leukemia, hairy cell leukemia and thyroid cancer.

Preferably, cancer according to the present invention may be selected from the group consisting of breast cancer, cervical cancer, colon cancer and lung cancer.

The condition according to the present invention may however also be a benign condition. For example the condition may be a benign tumour or the condition may be associated with hyperproliferation of connective tissue.

In one embodiment of the present invention the condition is associated with the presence of an undesirable large amount of a specific tissue or the presence of tissue that is undesirably active. Such undesirable tissue may not necessarily be malignant. It may be undesirable because it cause inconvenience, because its presence leads to a state of disease not related to cancer of the individual or for cosmetic reasons.

In particular, the condition may be selected from the group consisting of enlarged glands and hyperproductive glands. Glands according to the present invention may for example be selected from the group consisting of mammary glands, thyroid gland, prostate gland, pancreatic gland and salivary glands.

In addition, the condition may for example be overproduction of endocrine tissue, wherein it is desirable to remove part of or all the overproduced endocrine tissue. Other examples of conditions according to the present invention are autoimmune diseases. Autoimmune diseases are characterised by the presence of undesirable large amount of immune system cells directed against self-antigens of an individual.

Target cells according to the present invention are cells capable of being targeted, and which are desirable to target in order to treat a clinical condition. For example, the target cells may be indicative of said clinical condition or associated with said clinical condition. In one preferred embodiment the target cells are desirable to target in order to eliminate, inactivate or kill said cells.

Administration and Pharmaceutical Compositions

The treatment according to the present invention may be ameliorating treatment. Alternatively, the treatment may be curative or the treatment may be prophylactic.

The individual to receive treatment is any animal, however, preferably the individual is a human being.

Compounds of the kit-of-parts according to the present invention may be administered parenterally, that is by intravenous, intramuscular, subcutaneous intranasal, intrarectal, intravaginal or intraperitoneal administration. The subcutaneous and intramuscular forms of parenteral administration are generally preferred. Appropriate dosage forms for such administration may be prepared by conventional techniques. The compounds may furthermore be administered orally or topically. Additionally, the compounds may also be administered by inhalation, that is by intranasal and oral inhalation administration.

The delivery of the compounds of the invention to an individual can involve the use of e.g. targeting vehicles such as e.g. a posintro or a cationic ISCOM. Posintros or cationic ISCOMS can be any of the compounds described in the international patent application PCT/DK02/00229, which is hereby incorporated by reference in its entirety.

Preferably, the administration is by intravenous injection or the administration is by subcutaneous injection. In another preferred embodiment the administration is by perfusion.

In yet another preferred embodiment the administration is directly to the site of the target cells. For example the method may comprise administration of the antigen directly to the site of the target cells. Furthermore, for example the compounds according to the present invention may be injected directly into the site of the target cells.

In embodiments wherein the target cells are malignant cells of a solid tumour, the compounds may be injected directly into the tumour. Alternatively, the compounds may be applied directly to the site of the target cells with the aid of a genegun. Such an approach is in particular useful, when the condition is related to the skin, such as for example skin cancer or melanoma.

The compounds of the kit-of-parts according to the present invention may be administrated simultaneous as combined formulations or as separate formulations, or they may be administrated sequentially. Preferably, the parts of the kit-of-parts are administrated sequentially. More preferably, they are administrated such as the compound comprising the foreign immunogen is administrated prior to the compound comprising the foreign antigen, for example less than i day, such as at least 1 day, such as at least 3 days, for example at least 1 weeks, such as at least 2 weeks prior to administration of the compound comprising the foreign antigen.

In one embodiment of the present invention the treatment is administered more than once, such as twice, such as 3 times, for example 4 times, such as 5 times, for example 6 times, such as 7 times, for example 8 times, such as 9 times, for example 10 times, such as more than 10 times.

In one embodiment of the present invention the first part of the kit-of-parts may be administrated more than once, wherein the first part comprises a foreign immunogen.

Additionally, the second part of the kit-of-parts may be administrated more than once, wherein the second part comprises a foreign antigen.

It is also possible that one part of the kit-of-part is administered more frequently, than the other compound. For example the compound comprising the foreign immunogen may be administered once, such as twice, such as 3 times, for example 4 times, such as 5 times, for example 6 times, such as 7 times, for example 8 times, such as 9 times, for example 10 times, such as more than 10 times and the compound comprising the foreign antigen may be administered once, such as twice, such as 3 times, for example 4 times, such as 5 times, for example 6 times, such as 7 times, for example 8 times, such as 9 times, for example 10 times.

The compounds according to the present invention may be administrated more than once and more than one different immunogen and more than one different antigen may be administrated. Accordingly, it is possible to administrate a kit-of-parts comprising one set of immunogen/antigen, followed by administration of another kit-of-parts comprising another set of immunogen/antigen. For example, 1, such as 2, for example 3, such as 4, for example 5, such as 6, for example 7, such as 8, for example 9, such as 10, for example more than 10 different kit-of-parts comprising different sets of immunogen/antigen may be administrated.

Additionally, only different foreign antigens may be administrated, such as 1, such as 2, for example 3, such as 4, for example 5, such as 6, for example 7, such as 8, for example 9, such as 10, for example more than 10 different foreign antigens.

Each of the different foreign antigens and/or kit-of-parts may be administrated more than once, such as twice, such as 3 times, for example 4 times, such as 5 times, for example 6 times, such as 7 times, for example 8 times, such as 9 times, for example 10 times, such as more than 10 times.

The compounds of the kit-of-parts according to the invention may be administered with at least one other compound. The compounds may be administered simultaneously, either as separate formulations or combined in a unit dosage form, or administered sequentially.

In particular the compounds of the present invention may be administrated in combination with one or more different conventional therapies against cancer. Conventional therapies against cancer may be selected from the group consisting of surgical treatment, chemotherapy, radiation therapy, therapy with cytokines, hormone therapy, gene therapy, dendritic cell therapy or treatments using laser light.

Chemotherapy could include therapy using one or more drugs selected from: Melphalan, Carboplatin, Cyclophosphamid, Cisplatin, Ifosfamid, Chlorambucil, Lomustin Treosulfan, Temozolomid, Cytarabin, Azathioprin, Metothrexat, Fludarabinphosphat, Fluoruracil, Gemcitabin, Azathioprin, Cladribin, Podophyllotoksin, Etoposid, Topotecan, Vinkristin, Paclitaxel, Docetaxel, Vinblastin, Etoposid, Teniposid, Aclarubicin, Doxorubicin, Doxorubicin, Mitomycin, Mitoxantron, Idarubicin, Anon, Lenograstin, Filgrastim, Aldesleukin, Verteporfin, epirubicin, daunorubicin, valrubicin and adriamycinon.

The dosage requirements will vary with the particular drug composition employed, the route of administration and the particular individual being treated. Ideally, an individual to be treated by the present method will receive a pharmaceutically effective amount of the compound in the maximum tolerated dose, generally no higher than that required before drug resistance develops.

In general the daily parenteral dosage regimen may be about 0.001 to about 80 mg/kg of total body weight. It will also be recognised by one of skill in the art that the optimal quantity and spacing of individual dosages of a compound or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound or a pharmaceutically acceptable salt thereof given per day for a defined number of days, can be ascertained by those skilled in the art using conventional course of treatment determination tests.

The term “unit dosage form” as used herein refers to physically discrete units suitable as unitary dosages for human and animal individuals, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

Since the “effective level” is used as the preferred endpoint for dosing, the actual dose and schedule can vary, depending on interindividual differences in pharmacokinetics, drug distribution, and metabolism. The “effective level” can be defined, for example, as the blood or tissue level desired in the individual that corresponds to a concentration of one or more compounds according to the invention.

Pharmaceutical compositions containing a compound of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications.

Pharmaceutical acceptable salts of the compounds according to the present invention should also be considered to fall within the scope of the present invention. Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

Whilst it is possible for the compounds or salts of the present invention to be administered as the raw chemical, it is preferred to present them in the form of a pharmaceutical formulation. Accordingly, the present invention further provides a pharmaceutical formulation, for medicinal application, which comprises a compound of the present invention or a pharmaceutically acceptable salt thereof, as herein defined, and a pharmaceutically acceptable carrier therefor.

The compounds of the present invention may be formulated in a wide variety of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilisers, lubricants, suspending agents, binders, preservatives, wetting agents, tablet disintegrating agents, or an encapsulating material.

Preferably, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably containing from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa buffer, and the like. The term “preparation” is intended to include the formulation of the active compound with encapsulating material as carrier providing a capsule in which the active component, with or without carriers, is surrounded by a carrier, which is in association with it Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilised by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations, which are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavours, stabilising and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavours, stabilisers, buffers, artificial and natural sweeteners, dispersants, thickeners, solubilising agents, and the like.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilising and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used. In order to minimise or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The compounds of the invention can also be delivered topically. Regions for topical administration include the skin surface and also mucous membrane tissues of the vagina, rectum, nose, mouth, throat, and eye. Compositions for topical administration via the skin and mucous membranes should preferably not give rise to signs of irritation, such as swelling or redness.

The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, capsule, dry powder, syrup, balm or lozenge, for example. Methods for preparing such compositions are well known in the pharmaceutical industry.

The above topical compositions can be used individually or in any combination for any one or both of the method steps involving i) providing an individual with an immune response against a foreign immunogen not associated with a condition in said individual it is desirable to treat; and ii) administering to said individual a foreign antigen, which is capable of being recognised by the immune response raised against the immunogen and which is not associated with said condition. Any of the two above method steps can also be carried out by means other than topical administration, such as e.g. by conventional subcutaneous injection(s).

The compounds of the present invention may be formulated for topical administration to the epidermis as ointments, creams or lotions, or as a transdermal patch. Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturiser such as glycerol or an oil such as castor oil or arachis oil.

The pharmaceutical active compound described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like. According to one presently preferred hypothesis, transdermal administration of the compounds of the invention to an individual is believed to be particularly effective when using targeting vehicles such as e.g. a posintro or a cationic ISCOM as described herein above. Posintros or cationic ISCOMS can be any of the compounds described in the international patent application PCT/DK02/00229, which is hereby incorporated by reference in its entirety.

Transdermal delivery is accomplished by exposing a source of the active compound to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Such dosage forms can be made by dissolving, dispersing, or otherwise incorporating the pharmaceutical active compound in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%].

The compounds of the present invention may be formulated for vaginal administration. Pessaries, tampons, creams, gels, pastes, foams or sprays containing in addition to the active ingredient such carriers as are known in the art to be appropriate.

When desired, formulations can be prepared with enteric coatings adapted for sustained or controlled release administration of the active ingredient.

Pharmaceutical compositions usually comprise a carrier. Illustrative solid carrier include lactose, terra alba, sucrose, talc, gelatin, agar, pectin, acacia, magnesium stearate, stearic acid and the like. A solid carrier can include one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions, and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Illustrative liquid carriers include syrup, peanut oil, olive oil, water, etc. Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilisers, emulsifiers, buffers, preservatives, sweeteners, flavouring agents, suspending agents, thickening agents, colours, viscosity regulators, stabilisers or osmo-regulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carders are useful in sterile liquid form compositions for parenteral administration. The liquid carrier for pressurised compositions can be halogenated hydrocarbon or other pharmaceutically acceptable propellant. Liquid pharmaceutical compositions which are sterile solutions or suspensions can be utilised by, for example, intramuscular, intraperitoneal or subcutaneous injection. Sterile solutions can also be administered intravenously. The compound can also be administered orally either in liquid or solid composition form.

The carrier or excipient may include time delay material well known to the art, such as glyceryl monostearate or glyceryl distearate along or with a wax, ethylcellulose, hydroxypropylmethylcellulose, methylmethacrylate and the like. When formulated for oral administration, 0.01% Tween 80 in PHOSAL PG-50 (phospholipid concentrate with 1,2-propylene glycol, A. Nattermann & Cie. GmbH) has been recognised as providing an acceptable oral formulation for other compounds, and may be adapted to formulations for various compounds of this invention.

Immune Response

The immune response against the foreign immunogen may be present in the individual prior to the onset of treatment according to the present invention. For example such an immune response may have been generated following an infection of said individual.

In many cases however, the individual is not immune against the immunogen prior to the onset of treatment. Accordingly, the method according to the present invention include methods of raising an immune response to the immunogen in an individual.

Preferably, the method comprises the steps of

i) Providing an individual suffering from said condition; and

ii) Immunising said individual with the foreign immunogen; and

iii) Raising an immune response against the immunogen in said individual

Immunisation may be accomplished by any standard method known to the person skilled in the art. The immunogen may be comprised in a vaccine formulation as described herein above and adminstrated by any of the method described herein above.

In preferred embodiments the immunogen is administrated to the individual, internalised into cells of the individual, processed in said cells and displayed on the surface of said cells.

Once an immune response against a specific immunogen has been raised in an individual, the antigen may be administered to the individual and targeted to the cells of the individual desirable to target. The method of targeting antigen to target cells preferably comprises the steps of:

i) Internalising said antigen into said cells; and

ii) Displaying on the surface of said cells for example the antigen, such as a fragment of the antigen, such as a product of the antigen, for example a fragment of the product of the antigen.

Upon administration of the targeting vehicle of the kit-of-parts according to the present invention, preferably, the antigen is targeted to the target cell of the individual, and internalised into said target cells and displayed on the surface of said target cells.

The antigen may be directly displayed on the surface, however more frequently; fragments of the antigen or products of the antigen or fragment of products of the antigen are displayed.

The cytotoxic and/or inflammatory response according to the present invention may be mediated by any components of the immune system of the individual to be treated. Preferably, the cytotoxic and/or inflammatory response may be a cytolytic process or comprise a cytolytic process.

Preferably, however the cytotoxic and/or inflammatory response according to the present invention is mediated by cytotoxic T-cells. Such cytotoxic T-cells preferably express T-cell receptors that can associate with the antigen or fragments of the antigen or products of the antigen or fragments of products of the antigen according to the present invention.

However, the cytotoxic and/or inflammatory response may also be mediated by natural killer cells. In addition the cytotoxic and/or inflammatory response may for example be mediated by neutrophils or the cytotoxic and/or inflammatory response may be mediated eosinophils.

Furthermore, the cytotoxic and/or inflammatory response may be mediated by antibody-dependent cell-mediated cytotoxicity (ADCC) mechanisms. Such mechanisms preferably involve antibodies that can associate with the antigen or fragments of the antigen or products of the antigen or fragments of products of the antigen according to the present invention.

In one embodiment of the present invention, the cytotoxic and/or inflammatory response may be mediated by the innate immune system. For example the cytotoxic and/or inflammatory response may be mediated by the complement cascade. For example, the cytotoxic and/or inflammatory response may be mediated by the process of opsonisation by antibodies. For example, the cytotoxic and/or inflammatory response may be mediated by the process of opsonisation by one ore more components of the complement system.

In particular, the cytotoxic and/or inflammatory response may be mediated by of opsonisation of antibodies on the cellular surface of target cells by the classical complement pathway or the cytotoxic and/or inflammatory response may be initiated directly by the activation of complement factors, the alternative complement pathway.

Following the activation of one or both complement pathways the terminal membrane-attached complex may be formed, the target cell may be lysed and eliminated by said complex. In one preferred embodiment of the present invention the cytolytic process may result from the formation of a membrane-attack complex.

Antibodies that may activate the complement pathways according to the present invention, may for example be antibodies selected from the group consisting of IgG1, IgG2, IgG3 and IgM antibody isotypes.

Antibody-dependent cell-mediated cytotoxicity (ADCC) mechanisms may comprise other anti-cellular effectors than the complement pathways. For example antibodies bound to targeted cell may direct cytolytic activities of eosinophiles against the targeted cells. In such an embodiment the antibodies preferably are IgE.

Antibodies of any isotype may be present in the individual either by due to immunization using an immunogen as described herein above or by the passive transfer of heterologous antibodies to the individual to be treated. Heterologous antibodies may be derived from any suitable sources known to the person skilled in the art.

In addition, the cytotoxic and/or inflammatory response may be mediated by a combination of two or more mechanisms mentioned herein above. For example it may be mediated by two or more selected from the group consisting of cytotoxic T-cells, natural killer cells, neutrophils, eosinophils, antibody-dependent cell-mediated cytotoxicity and cytolytic mechanisms.

It is possible to control the cytotoxic and/or inflammatory response by selecting a suitable combination of foreign immunogens and foreign antigens. In particular, the duration of the cytotoxic and/or inflammatory response may be controlled according to the specific needs. For applications wherein undesirable large amounts of tissues is to be removed the cytotoxic and/or inflammatory responses should preferably only be short, whereas for other applications it may be desirable with a longer lasting response.

A short cytotoxic and/or inflammatory response may last for example for less than 1 hour, such as less than 2 hours, for example less than 3 hours, such as less than 6 hours, for example less than 12 hours, such as less than 24 hours, for example less than 48 hours.

A longer lasting cytotoxic and/or inflammatory response may last for example for more than 1 day, such as more than 2 days, for example more than 1 week, such as more than 2 weeks, for example more than 1 month, such as more than 3 months, for example more than 1 year, such a more than 3 years, for example more than 5 years, such as more than 10 years, for example in principle throughout life.

Examples of foreign immunogens and/or foreign antigens, which may result in long lasting responses, are foreign immunogens and/or foreign antigens, which are derived from herpes simplex virus type 1, herpes simplex virus type 2, Varicelle-Zoster virus, Epstein-Barr virus, Cytomeglovirus, human herpes virus-6, human herpes virus-7, human herpes virus-8, influenzavirus, rubella, polio virus, polio vaccine virus (Sabin virus), vaccinia virus.

Examples of foreign immunogens and/or foreign antigens, which may result in short responses are foreign immunogens and/or foreign antigens which comprise ovalubumin, keyhole limpet hemocyanin, sperm-whale myoglobin or fragments thereof.

It is of significant importance to note the cytotoxic and/or inflammatory response according to the present invention are not directed against antigens naturally present on the target cells, but rather is directed against foreign antigens, which are not natively associated with the target cells.

Accordingly, the cytotoxic and/or inflammatory responses according to the present invention are not directed against tumour associated antigens, associated with a given tumour to be targeted.

Furthermore, the methods disclosed herein preferably, do not involve epitope spreading such as the cytotoxic and/or inflammatory responses according to the present invention are not redirected to involve responses against antigens naturally associated with the target cells.

### EXAMPLE

Model tumors are established by injection of 5×10⁶ EL4 (a thymoma call line) subcutaneously in syngeneic C57BL/6 mice.

Eight and four weeks prior to injection of EL4 cells mice were immunized with a DNA vaccine encoding hepatitis B virus S antigen (gWIZ-HbsAg) purchased from Aldevron, N.D., in order to establish a strong cytotoxic T-cell response against the S antigen. Mice received 100 μg of the plasmid vaccine intramuscularly at each time point.

Three weeks after the injection of EL4 cells solid tumors can be observed at and around the site of injection. At this time gWIZ-HbsAg plasmid (approx. 20 μg) formulated with was injected into selected well-localized tumors and the tumor progression was measured on a daily basis by measuring the diameter and the height of the individual tumors and a semi quantitative mean tumor surface area was calculated for each measurement.

Control animals that did not receive the plasmid vaccine prior to EL4 cell injection was used as reference and the percentage of tumor reduction/growth was calculated for each individually localized tumor.

Tumors of animals that was immunized with gWIZ-HbsAg prior to EL4 cell inoculation and was injected at the tumor site with the same plasmid showed a reduced growth compared to non-treated tumors and tumors of non-vaccinated animals. However, the variation of tumor reduction/growth inhibition varied. It is concluded that the dual treatment (vaccination/tumor injection) has some effect on predicted tumor growth, compared to non-treated tumors.

1. A kit of parts comprising
i) A foreign immunogen essentially consisting of one or more polypeptide and/or one or more peptides; or
one or more nucleic acids sequences encoding a polypeptide and/or a peptide; and
ii) A targeting complex comprising
a) a targeting vehicle selected from the group consisting of cationic ISCOMs, ISCOMs, liposomes, lipid vesicles, niosomes, cochleates, biodegradable microspheres, nanoparticles, hydrogels and microcrystals, and
b) a foreign antigen, which can be recognised by an immune response raised against the immunogen, wherein said foreign antigen essentially consists of one or more polypeptide and/or one or more peptides or one or more nucleic acids sequences encoding a polypeptide or a peptide
and wherein the antigen and the immunogen are the same, or
the antigen is a fragment og the immunogen; or
the immunogen is a fragment of the antigen; or
the antigen comprises a nucleic acid encoding the immunogen or a fragment thereof;
or the immunogen comprises a nucleic acid encoding the antigen or a fragment thereof.

2. The kit-of-parts according to claim 1, wherein the immunogen comprises a polypeptide and/or peptide.

3. The kit-of-parts according to claim 1, wherein the immunogen comprises a glucosylated polypeptide and/or peptide.

4. The kit-of-parts according to claim 1, wherein the immunogen comprises a nucleic acid sequence.

5. The kit-of-parts according to claim claim 1, wherein the immunogen comprises a peptide or a polypeptide chemically linked to a lipid moiety.

6. The kit-of-parts according to claim 1, wherein the immunogen comprises a peptide or a polypeptide chemically linked hapten linked to a carrier molecule.

7. The kit-of-parts according to claim 1, wherein the immunogen is a multivalent immunogen.

8. The kit-of-parts according to claim 4, wherein the nucleic acid sequence encode a polypeptide and/or peptide.

9. The kit-of-parts according to any of claims 2 and 8, wherein the polypeptide is foreign to the human body.

10. The kit-of-parts according to claim 1, wherein the immunogen is derived from a virus.

11. The kit-of-parts according to claim 1, wherein the immunogen is derived from a virus selected from the group consisting of influenza viruses, herpes viruses, morbili viruses, myxo- and paramyxoviruses, flaviviruses, papillomaviruses and hepatitis viruses.

12. The kit-of-parts according to claim 1, wherein the immunogen is derived from a bacteria.

13. The kit-of-parts according to claim 1, wherein the immunogen is derived from a parasite.

14. The kit-of-part according to claim 1, wherein the immunogen is comprised within a vaccine formulation.

15. The kit-of-part according to claim 14, wherein the vaccine formulation furthermore comprises an adjuvant.

16. The kit-of-part according to claim 14, wherein the vaccine formulation furthermore comprises a carrier.

17. The kit-of-part according to claim 14, wherein the vaccine formulation furthermore comprises a biological active component.

18. The kit-of-parts according to claim 17, wherein the biological active component is selected from the group consisting of cytokines and chemokines.

19. The kit-of-parts according to claim 1, wherein the immunogen is associated with a targeting vehicle selected from the group consisting of cationic ISCOMs, ISCOMs, liposomes, lipid vesicles, niosomes, cochleates, biodegradable microspheres, nanoparticles, hydrogels and microcrystals.

20. The kit-of-parts according to claim 19, wherein the targeting vehicle is an ISCOM.

21. The kit-of-parts according to claim 19, wherein the targeting vehicle is an ISCOM comprising a net positive charge at pH 7.0.

22. The kit-of-parts according to claim 1 wherein the antigen comprises a polypeptide and/or a peptide.

23. The kit-of-parts according to claim 1, wherein the antigen comprises a glucosylated polypeptide and/or peptide.

24. The kit-of-parts according to claim 1, wherein the antigen is a nucleic acid sequence.

25. The kit-of-parts according to claim 1, wherein the antigen is a multivalent antigen.

26. The kit-of-parts according to claim 1, wherein the antigen is a hapten linked to a carrier molecule.

27. The kit-of-parts according to claim 24, wherein the nucleic acid sequence encodes a polypeptide and/or a peptide.

28. The kit-of-parts according to any of claims 22 and 27, wherein the polypeptide is foreign to the human body.

29. The kit-of-parts according to claim 1, wherein the antigen is derived from a virus.

30. The kit-of-parts according to claim 1, wherein the antigen is derived from a virus selected from the group consisting of influenza viruses, herpes viruses, morbili viruses, myxo- and paramyxoviruses, flaviviruses, papillomaviruses and hepatitis viruses.

31. The kit-of-parts according to claim 1, wherein the antigen is derived from a bacteria.

32. The kit-of-parts according to claim 1, wherein the antigen is derived from a parasite.

33. The kit-of-parts according to any of claims 1 to 32, wherein the antigen and the immunogen are the same.

34. The kit-of-parts according to any of claims 1 to 32, wherein the antigen is a fragment of the immunogen.

35. The kit-of-parts according to any of claims 1 to 32, wherein the immunogen is a fragment of the antigen.

36. The kit-of-parts according to any of claims 1 to 32, wherein the antigen mimics the immunogen.

37. The kit-of-parts according to claim 1, wherein the vehicle comprises a posintro.

38. The kit-of-parts according to claim 1, wherein the vehicle comprises an ISCOM.

39. The kit-of-parts according to claim 1, wherein the vehicle comprises a liposome.

40. The kit-of-parts according to claim 1, wherein the vehicle comprises a biodegradable microsphere.

41. The kit-of-part according to claim 1, wherein the vehicle comprises an encapsulation system.

42. The kit-of-part according to claim 1, wherein the vehicle comprises a cochleate.

43. The kit-of-part according to claim 1, wherein the vehicle comprises a nanoparticle.

44. The kit-of-part according to claim 1, wherein the vehicle comprises a hydrogel.

45. The kit-of-part according to claim 1, wherein the vehicle comprises a microcrystal.

46. The kit-of-part according to claim 1, wherein the vehicle further comprises a lipid, which can associate with lipid rafts of the target cells.

47. The kit-of-parts according to claim 1, wherein the vehicle further comprises a specific binding partner.

48. The kit-of-part according to claim 1, wherein the vehicle further comprises a specific binding partner, which is capable of being internalised.

49. The kit-of-part according to claim 1, wherein the vehicle further comprises a specific binding partner, which can be taken up by target cells by receptor mediated endocytosis.

50. The kit-of-part according to claim 1, wherein the vehicle further comprises a specific binding partner, wherein the specific binding partner is a vitamin.

51. The kit-of-part according to claim 1, wherein the vehicle further comprises a specific binding partner, wherein the specific binding partner is folic acid.

52. The kit-of-parts according to claim 1, wherein the targeting complex further comprises a biologically active component.

53. The kit-of-parts according to claim 52, wherein the biologically active component is selected from the group consisting of cytokines and chemokines and nucleic acid sequences encoding cytokines and chemokines.

54. The kit-of-parts according to claim 52, wherein the biologically active component is a compound capable of inducing apoptosis in the targeted cell.

55. The kit-of-parts according to claim 52, wherein the biologically active component is a member of the Pro-Apoptotic Bcl-2 Family of proteins.

56. The kit-of-parts according to claim 52, wherein the biologically active component is a subunit of the Apoptasome Complex.

57. The kit-of-parts according to claim 52, wherein the biological active component is a Caspase.

58. The kit-of-parts according to claim 1, wherein the targeting complex further comprises a cytostatic compound.

59. The kit-of-parts according to claim 1. wherein the kit-of-part comprise more than one immunogen.

60. The kit-of-part according to claim 1, wherein the kit-of-parts comprise more than one antigen.

61. A pharmaceutical composition comprising the kit-of-parts according to any of the claims 1 to 60, together with pharmaceutical acceptable carriers.

62. A method of treatment of a condition, which is characterised by the presence of cells capable of being targeted, which are desirable to eliminate in order to treat the clinical conditions, in an individual in need thereof comprising the steps of
i) Providing an individual suffering from said condition; and
ii) Immunising said individual with a foreign immunogen essentially consisting of one or more polypeptide and/or one or more peptides or one or more nucleic acids sequences encoding a polypeptide or a peptide; and
iii) Raising an immune response against the immunogen in said individual
iv) Administering to said individual a foreign antigen, which is capable of being recognised by the immune response raised against the immunogen and which is not associated with said condition, wherein said foreign antigen essentially consists of one or more polypeptide and/or one or more peptides or one or more nucleic acids sequences encoding a polypeptide or a peptide; and
v) Targeting said antigen to the cells of said individual, which are desirable to target; and
vi) Enabling a cytotoxic and/or inflammatory response against said foreign antigen in the individual,
wherein the antigen and the immunogen are the same, or
the antigen is a fragment og the immunogen; or
the immunogen is a fragment of the antigen; or
the antigen comprises a nucleic acid encoding the immunogen or a fragment thereof;
or the immunogen comprises a nucleic acid encoding the antigen or a fragment thereof.

63. The method according to claim 62, wherein step v) furthermore comprises the steps of
i) Internalising said antigen into said cells; and
ii) Displaying on the surface of said cells for example the antigen, such as a fragment of the antigen, such as a product of the antigen, for example a fragment of the product of the antigen.

64. The method according to claim 62, wherein step iii) comprises administration of the antigen directly to the site of the target cells.

65. A method of treatment of a condition, which is characterised by the presence of cells, which are desirable to eliminate, in an individual in need thereof comprising administering to said individual the kit-of-parts according to any of the claims 1 to 60, and thereby enabling a cytotoxic and/or inflammatory response against the foreign antigen.

66. The method according to claim 65, wherein the antigen is targeted to the target cell of the individual, and internalised into said target cells and displayed on the surface of said target cells.

67. The method according to claim 65, wherein the parts of the kit-of-parts are administrated sequentially.

68. The method according to claim 65, wherein the part i) of the kit-of-parts is administrated more than once.

69. The method according to claim 65, wherein the part ii) of the kit-of-parts is administrated more than once.

70. The method according to any of the claims 62 and 65, wherein the condition is cancer.

71. The method according to any of the claims 62 and 65, wherein the condition is cancer selected from the group consisting of breast cancer, cervical cancer, colon cancer and lung cancer.

72. The method according to any of the claims 62 and 65, wherein the condition is a benign tumour.

73. The method according to any of the claims 62 and 65, wherein the condition is associated with hyperproliferation of connective tissue.

74. The method according to any of the claims 62 and 65, wherein the condition is overproduction of endocrine tissue.

75. The method according to any of the claims 62 and 65, wherein the condition is an autoimmune disease.

76. The method according to any of the claims 62 and 65, wherein the condition is selected from the group consisting of enlarged glands and hyperproductive glands.

77. The method according to claim 76, wherein the glands are selected from the group consisting of mammary glands, thyroid gland, prostate gland, pancreatic gland and salivary glands.

78. The method according to any of the claims 62 and 65, wherein the treatment is ameliorating.

79. The method according to any of the claims 62 and 65, wherein the treatment is curative.

80. The method according to any of the claims 62 and 65, wherein the treatment is prophylactic.

81. The method according to any of the claims 62 and 65, wherein administration is by intravenous injection.

82. The method according to any of the claims 62 and 65, wherein administration is topical and involves a transdermal patch.

83. The method according to any of the claims 62 and 65, wherein the administration is by subcutaneous injection.

84. The method according to any of the claims 62 and 65, wherein the administration is by perfusion.

85. The method according to any of the claims 62 and 65, wherein the administration is directly to the site of the target cells.

86. The method according to any of the claims 62 and 65, wherein the administration is more than once, such as twice, such as 3 times, for example 4 times, such as 5 times, for example 6 times, such as 7 times, fro example 8 times, such as 9 times, for example 10 times, such as more than 10 times.

87. The method according to any of the claims 62 and 65, wherein the administration is more than once and wherein more than one different immunogen and/or more than one different antigen is administrated.

88. The method according to any of the claims 62 and 65, wherein the treatment furthermore comprise one or more different conventional therapies against cancer selected from the group consisting of surgical treatment, chemotherapy, radiation therapy, therapy with cytokines, hormone therapy, gene therapy, dendritic cell therapy or treatments using laser light.

89. A method of non-invasive surgery comprising the methods according to any of the claims 62 to 88.

90. A use of the kit-of-part according to any of the claims 1 to 60, together with a pharmaceutically acceptable carrier for the preparation of a medicament for the treatment of a condition, which is characterised by the presence of cells, which are desirable to eliminate.

91. The use according to claim 90, wherein the condition is cancer.

92. The use according to claim 90, wherein the condition is cancer selected from the group consisting of breast cancer, cervical cancer, colon cancer and lung cancer.

93. The use according to claim 90, wherein the condition is a benign tumour.

94. The use according to claim 90, wherein the condition is associated with hyperproliferation of connective tissue.

95. The use according to claim 90, wherein the condition is overproduction of endocrine tissue.

96. The use according to claim 90, wherein the condition is an autoimmune disease.

97. The use according to claim 90, wherein the condition is enlarged glands.

98. The use according to claim 97, wherein the glands are selected from the group consisting of mammary glands, thyroid gland, prostate gland, pancreatic gland and salivary glands.
